(19) United States
(12) Patent Application Publication
(10) Pub. No.: US 2026/0258094 A1
(43) Pub. Date: Sep. 3, 2026

(54) MODIFIED BACTERIAL MICROCOMPARTMENT SHELL PROTEINS

(71) Applicant: THE REGENTS OF THE UNIVERSITY OF CALIFORNIA, Oakland, CA (US)

(72) Inventors: Henning KIRST, Berkeley, CA (US); Eric J. YOUNG, Berkeley, CA (US); John STEELE, Berkeley, CA (US); Cheryl A. KERFELD, Walnut Creek, CA (US)

(21) Appl. No.: 19/359,587

(22) Filed: Oct. 15, 2025

Related U.S. Application Data
(60) Provisional application No. 63/708,135, filed on Oct. 16, 2024.

(51) Int. Cl. C07K 14/195; C12N 15/62

(52) U.S. Cl. CPC C07K 14/195; C12N 15/62; C07K 2319/00

(57) ABSTRACT

The present invention provides for a fusion protein comprising (1) a bacterial microcompartment (BMC) shell protein, and (2) a first component of a specific-binding pair linked to the BMC shell protein such that the first component faces (i) a lumen (inside) side, or (ii) outside of a BMC shell formed incorporating the fusion protein; wherein the first component is capable of forming a stable or irreversible interaction with a second component of the specific-binding pair, and the second component comprises a cargo or compound capable of generating an output directly coupled to the molecular permeability of the BMC shell.

### CROSS-REFERENCE TO RELATED APPLICATIONS

This application claims priority to U.S. Provisional Patent Application Ser. No. 63/708,135, filed Oct. 16, 2024, which are hereby incorporated by reference.

### STATEMENT OF GOVERNMENTAL SUPPORT

The invention was made with government support under Contract Nos. DE-AC02-05CH11231 awarded by the U.S. Department of Energy, and Grant No. 5R01AI114975-08 awarded by the National Institutes of Health. The government has certain rights in the invention.

### REFERENCES TO SUBMISSION OF A SEQUENCE LISTING

The instant application contains a Sequence Listing which has been submitted electronically in XML format and is hereby incorporated by reference in its entirety. Said XML copy, created on May 8, 2026, is named “2025-005-02_Sequence_Listings”, and is 15,145 bytes in size.

### FIELD OF THE INVENTION

The present invention is in the field of bacterial microcompartments (BMCs).

### BACKGROUND OF THE INVENTION

Compartmentalization is a fundamental characteristic of life. In eukaryotes, compartment membranes are lipid-based. In contrast, in bacteria, entirely protein-based organelles known as bacterial microcompartments (BMCs) are widespread 1,2. The “membranes” of BMCs are composed from two protein domain families: pfam00936 or pfam03319 1,3,4. BMC-Hexamer (BMC-H) proteins contain a single pfam00936 domain and form hexamers 5. BMC-Tandem (BMC-T) proteins consist of a tandem fusion of pfam00936 domains 6 and form trimers. These oligomers form hexagonally shaped tiles that form the facets of a polyhedral shell 4,7-14. Pentamers that cap the vertices of the shell are formed by BMC-Pentamer (BMC-P) proteins that contain a single pfam03319 domain 15. It is hypothesized that spatial proximity of enzymes in the core in conjunction with surrounding shell proteins benefits pathways via enzyme and substrate colocalization in a defined volume, diminishing cross-talk with metabolism in the bulk cytosol, while confining toxic intermediates within the BMC 1,16. The selective permeability properties of a BMC shell is accepted to play an essential role in supporting these benefits.

Routes for molecules to cross BMC shells are hypothesized to include shell compositional changes 17,18, travel through a shell protein's centrally located pore 5, and/or shell leakiness 5. Simulations and structures of BMC shell proteins support that BMC shell protein pores act as a conduit for metabolites and cofactor sized molecules 5,6,19-24. Also, in vivo or in vitro characterized BMCs with amino acid substitutions of pore lining residues in shell proteins, or removal of the shell proteins entirely, qualitatively suggests that pore structure (and the shell itself) influences molecular permeability 25-37. Another way BMC permeability appears modifiable is by altering the complement of shell proteins in the shell at a particular life-cycle or environmental cues 38-41. Indeed, study of a synthetic BMC shell system with programmed loading of a permeability probe supported that larger molecular cargos (about 40 Å diameter) enter shells when BMC-P vertices of a shell are left deliberately “uncapped” 42. When BMC shells are intact and capped, studies with fluorescent small-molecules suggest that a shell's lumen is still chemically accessible to about 10 Å diameter molecules 42,43. This supports BMC shell protein pores (and/or other non-specific pathways) likely dictate molecular permeability properties of a shell. High resolution data sets are still needed to characterize the extent to which a BMC shell acts as a diffusion barrier for small molecules. These insights are necessary to improve fundamental understanding of BMCs in microbiomes, pathogenesis, and carbon cycling and to advance BMC shells as bionanotechnologies.

Fiedler et al. (“RNA-Directed Packaging of Enzymes within Virus-like Particles,” Angewandte Chemie, 49 (50): 9648-9651, 2010) disclose packaging His-tagged peptidase E, firefly luciferase (Luc), or a thermostable mutant of Luc (tsLuc) inside Qβ virus-like particles (VLPs).

### SUMMARY OF THE INVENTION

The present invention provides for a fusion protein comprising (1) a bacterial microcompartment (BMC) shell protein, and (2) a first component of a specific-binding pair linked to the BMC shell protein such that the first component faces (i) a lumen (inside) side, or (ii) outside of a BMC shell formed incorporating the fusion protein; wherein the first component is capable of forming a stable or irreversible interaction with a second component of the specific-binding pair, and the second component comprises a cargo or compound capable of generating an output directly coupled to the molecular permeability of the BMC shell.

In some embodiments, the fusion protein comprises (1) a bacterial microcompartment (BMC) shell protein comprising one or more subunit, and (2) a first component of a specific-binding pair, operably linked to the BMC shell protein such that the first component faces (i) a lumen (inside) side, or (ii) outside of a BMC shell formed incorporating the fusion protein and the fusion protein does not disrupt or prevent the folding of the BMC shell protein or the ability of the BMC shell protein to integrate with other BMC shell proteins into a BMC shell; wherein the first component is capable of forming a stable or irreversible interaction with a second component of the specific-binding pair; wherein the second component comprises a cargo or compound capable of generating an output directly coupled to the molecular permeability of the BMC shell.

In some embodiments, the BMC shell lacks pentamers containing about 5 nm vertex gaps (i.e. “uncapped” configuration). In some embodiments, the BMC shell structures re prepared in PyMol from PDB structure 5V74. In some embodiments, the BMC-T1 shell protein is fused to a SpyTag/SnoopTag. In some embodiments, a reciprocally-tagged-catcher cargo is covalently tethered, such that the cargo is in the lumen of the assembled BMC shell. In some embodiments, the cargo comprises an enzyme, such as an enzyme capable of detecting the presence of ATP, such as a luciferase. In some embodiments, the cargo comprises an enzyme capable of detecting the presence of ATP which can be used to evaluate the permeability of ATP to a BMC shell. In some embodiments, the second component comprises an enzyme, such as an enzyme capable of detecting the presence of ATP, such as a luciferase. In some embodiments, the second component comprises an enzyme capable of detecting the presence of ATP which can be used to evaluate the permeability of ATP to a BMC shell.

In some embodiments, the stable or irreversible interaction is a spontaneous formation of a covalent bond. In some embodiments, the covalent bond is an isopeptide bond (which may for example occur between a lysine and an asparagine residue in an appropriate environment).

In some embodiments, the ability of the BMC shell protein to integrate with other BMC shell proteins into a BMC shell is the ability to assemble in vitro or in vivo.

In some embodiments, the BMC shell protein subunit is BMC-H, BMC-T, or BMC-P. In some embodiments, the BMC-T shell protein subunit is BMC-T1, BMC-T2, or BMC-T3.

In some embodiments, the first component of a specific-binding pair is a peptide. In some embodiments, the second component of the specific-binding pair is a peptide. In some embodiments, the first component of the specific-binding pair is flanked by a sequence of amino acids that adequately present the specific component to its cognate binding partner. In some embodiments, the sequence of amino acids that adequately present the specific component to its cognate binding partner is at least about one, two, three, four, five, or six glycine-serine linkers. In some embodiments, the binding between the first component and the second component of a specific-binding pair has an association constant of equal to or greater than about 10⁸ M⁻¹. Whether an amino acid residue of a BMC shell protein is facing inside or outside can be determined by a number of different methods. In one method, the BMC shell protein is purified and crystallized, and its three-dimensional configuration determined and compared to the structure of the shell protein in a complete shell. The amino acid residues between secondary structure elements, such as between two alpha-helices, two beta-strands, or an alpha-helix and a beta-strand are generally amenable for insertions. The residues on the concave surface of the BMC shell protein are facing inside and the amino acid residues on the convex surface of the BMC shell protein are facing outside and insertions in the loop regions of the two sides will face the respective direction. In another method, the primary structure of a BMC shell protein is compared to the primary structure of a BMC shell protein having high amino acid sequence identity which three-dimensional configuration is known. The inside and outside amino acid residues from the latter can be inferred on the former.

For fusion proteins where the first component faces a lumen of the BMC shell protein, the first component is operably linked to within or adjacent to any amino acid residue of a BMC shell protein that faces the lumen or inside of a BMC shell, when the BMC shell protein is incorporated in the BMC shell. In some embodiments, the BMC shell protein is BMC-H/HO-5815. In some embodiments, when the BMC-T shell protein subunit is BMC-T1, and the first component is operably linked or inserted on the lumenal side between alpha-helix two and beta-strand four of BMC-T1. The first component can be operably linked or inserted on the lumenal side, or inside, of any location of any BMC shell protein subunit and tested using the methods described herein to determine if the first component faces a lumen of the BMC shell protein and does not disrupt or prevent the folding of the BMC shell protein or the ability of the BMC shell protein to integrate with other BMC shell proteins into a BMC shell.

For fusion proteins where the first component faces outside of the BMC shell protein, the first component is operably linked to the N-terminus, C-terminus, or within or adjacent to any amino acid residue of a BMC shell protein that faces outside (i.e. on the external surface) of a BMC shell, when the BMC shell protein is incorporated in the BMC shell. In some embodiments, BMC shell protein is BMC-H/HO-5815. The first component can be operably linked or inserted on the outside, of any location of any BMC shell protein subunit and tested using the methods described herein to determine if the first component faces outside of the BMC shell protein and does not disrupt or prevent the folding of the BMC shell protein or the ability of the BMC shell protein to integrate with other BMC shell proteins into a BMC shell.

In some embodiments, the second component is linked to a “cargo”, which can be any polypeptide or compound of interest. In some embodiments, the cargo can a pharmaceutical or therapeutic agent. In some embodiments, the second component, or the cargo, is an antigen capable of causing an immune response in a subject, wherein optionally the first component faces the outside. In some embodiments, the second component, or the cargo, is an antigen capable of causing an immune response in a subject such that the subject is immunized to a pathogen that comprises or presents the antigen. In some embodiments, the cargo comprises an enzyme, such as an enzyme capable of detecting the presence of ATP, such as a luciferase. In some embodiments, the cargo comprises an enzyme capable of detecting the presence of ATP which can be used to evaluate the permeability of ATP to a BMC shell.

In some embodiments, the first component is inserted into a shell protein at a location facing the lumen of the shell and that does not disrupt the global folding of the protein or its ability to integrate into BMC shells. The first component can also be appended to the N- or C-termini of shell proteins which project outwards from the shell in their normal context or inwards in a permuted version of the BMC-H shell protein. Separately, proteins or inorganic materials that are to be encapsulated or adhered (referred to hereafter as “cargo”) are fused to the first or second component, such as a short (about 13 residues) amino acid sequence (such as the amino acid sequence called “SpyTag”) When co-present either in vivo or in vitro, the fusion protein (through the first component) and second component associate and form an isopeptide bond that covalently links the two polypeptides.

In some embodiments, there is the insertion of a split bacterial adhesin domain called “SpyCatcher” into a shell protein at a location facing the lumen of the shell and that does not disrupt the global folding of the protein or its ability to integrate into BMC shells. In some embodiments, the SpyCatcher domain is also appended to the N- or C-termini of shell proteins which project outwards from the shell in their normal context. In some embodiments, separately, the proteins or inorganic materials that are to be encapsulated or adhered (referred to hereafter as “cargo”) are fused to a short (about 13 residues) amino acid sequence called “SpyTag.” In some embodiments, when co-present either in vivo or in vitro, the SpyCatcher-shell protein and SpyTag-cargo associate and form an isopeptide bond that covalently links the two polypeptides.

In some embodiments, the first component comprises an amino acid sequence of any of the sequence taught herein. In some embodiments, the binding partner comprises an amino acid sequence of any of the sequence taught herein. In some embodiments, the first component or second component comprises an amino acid sequence as described in U.S. Pat. No. 9,547,003 (hereby incorporated by reference), and Zakeri, et al. Proc. Natl. Acad. Sci. USA, 109(12): E690-E697, 2012. In some embodiments, the first component is a receptor site and the second component is a cofactor or compound that binds to the receptor site, wherein optionally the first component faces the outside. In some embodiments, the SpyTag/SpyCatcher sequence is replaced with an “AviTag” which is biotinylated by a BirA protein (either in vivo or in vitro). This can then recruit other “avidin” type proteins that bind very tightly but non-covalently to the biotin cofactor. When AviTagged proteins are used, BMC shells can be formed, and avidins are capable of binding to them.

In some embodiments, the first component faces the outside and is an affinity handle for purifying or isolating the BMC shell. In some embodiments, the second component is bound to a solid support, such as a resin, such as a resin bead or a resin column.

In some embodiments, the first component is a peptide tag and the second component is a binding partner that specifically binds to the peptide tag. In some embodiments, the first component/second component, or peptide tag/binding partner, pairs with stable or irreversible interactions by adapting a feature of amino acid chemistry, namely the spontaneous formation of isopeptide bonds (which may for example occur between a lysine and an asparagine residue in an appropriate environment).

Examples of known proteins capable of spontaneously forming one or more isopeptide bonds include Spy0128 (Kang et al, Science, 2007, 318 (5856), 1625-8), Spy0125 (Pointon et al, J. Biol. Chem., 2010, 285 (44), 33858-66) and FbaB (Oke et al, J. Struct Funct Genomics, 2010, 11 (2), 167-80) from Streptococcus pyogenes, Cna of Staphylococcus aureus (Kang et al, Science, 2007, 318 (5856), 1625-8), the ACE19 protein of Enterococcus faecalis (Kang et al, Science, 2007, 318 (5856), 1625-8), the BcpA pilin from Bacillus cereus (Budzik et al, PNAS USA, 2007, 106 (47), 19992-7), the minor pilin GBS52 from Streptococcus agalactiae (Kang et al, Science, 2007, 318 (5856), 1625-8), SpaA from Corynebacterium diphtheriae (Kang et al, PNAS USA, 2009, 106 (40), 16967-71), SpaP from Streptococcus mutans (Nylander et al, Acta Crystallography Sect F Struct Biol Cryst Comm, 2011, 67 (Pt1), 23-6), RrgA (Izore et al, Structure, 2010, 18 (1), 106-15), RrgB (El Mortaji et al, J. Biol. Chem., 2010, 285 (16), 12405-15) and RrgC (El Mortaji et al, J. Biol. Chem., 2010, 285 (16), 12405-15) from Streptococcus pneumoniae, SspB from Streptococcus gordonii (Forsgren et al, J Mol Biol, 2010, 397 (3), 740-51). As discussed above, any of these proteins may hence be used in the present invention to develop a peptide tag/binding partner pair.

In some embodiments, the peptide tag and binding partner pair are wherein (a) said peptide tag is a peptide fragment of an isopeptide protein, said tag having a length of at least 5 amino acids but no more than 50 amino acids, and comprises a first reactive residue involved in formation of an intramolecular isopeptide bond in an isopeptide protein, wherein said peptide tag is either unconjugated or is conjugated to a heterologous protein or peptide or to another molecule and wherein said isopeptide protein is selected from the group consisting of (i) major pilin protein Spy0128 from Streptococcus pyogenes as set forth in U.S. Pat. Nos. 11,130,788 and 12,071,455 (which are hereby incorporated by reference) or a protein with at least 95% identity thereto capable of spontaneously forming an isopeptide bond, or (ii) FbaB from S. pyogenes as set forth in U.S. Pat. Nos. 11,130,788 and 12,071,455 (which are hereby incorporated by reference), or a protein with at least 80% identity thereto capable of spontaneously forming an isopeptide bond; (b) said binding partner (i) comprises a different fragment of an isopeptide protein as set forth in (a) (i) or (a) (ii) wherein said fragment is at least 20 amino acids in length and (ii) comprises a second reactive residue involved in the isopeptide bond in said isopeptide protein, wherein the binding partner does not include the first reactive residue of the peptide tag; and (c) said peptide tag and binding partner are capable of binding to each other and forming an isopeptide bond between the first and second reactive residues.

The present invention also provides for a BMC comprising encapsulating a compound of interest.

The present invention also provides for a nucleic acid encoding the fusion protein of the present invention.

The present invention also provides for a host cell comprising the nucleic acid encoding the fusion protein of the present invention capable of expressing the fusion protein.

The present invention also provides for a two-dimensional sheet of BMC proteins comprising the fusion protein of the present invention. In some embodiments, the two-dimensional sheet of BMC proteins further comprises a corresponding second component, in a stable or irreversible interaction with of the first component of the fusion protein. In some embodiments, the two-dimensional sheet of BMC proteins comprises two or more different fusion protein wherein each fusion protein independently has a different first component that associates with a different corresponding second component. In some embodiments, each corresponding second component is associated or linked, such as covalently bound, to a different cargo or a different two-dimensional sheet of BMC proteins.

The present invention also provides for methods for making or using the fusion protein, BMC, nucleic acid, or host cell of the present invention.

The present invention also provides for a method for making a fusion protein of the present invention, the method comprising: (a) introducing a nucleic acid encoding the first component into the nucleic acid encoding a BMC shell protein to produce a nucleic acid encoding the fusion protein of the present invention, and (b) optionally expressing the fusion protein and optionally other BMC shell proteins such that the fusion protein and other shell proteins self-assemble into a BMC shell, wherein the first component faces (i) a lumen (inside) side, or (ii) outside of a BMC shell formed incorporating the fusion protein.

The present invention also provides for a method for purifying or isolating a BMC shell of the present invention, the method comprising: (a) introducing a nucleic acid encoding the first component into the nucleic acid encoding a BMC shell protein to produce a nucleic acid encoding the fusion protein of the present invention, wherein the first component faces the outside of the BMC shell protein, (b) optionally expressing the fusion protein and optionally other BMC shell proteins such that the fusion protein and the other shell proteins self-assemble into a BMC shell comprising the first component on the outside of the BMC shell, (c) contacting a composition comprising the BMC shell comprising the first component with the second component, such that the first component and the second component specifically bind to form a BMC shell-solid support complex, wherein the second component is bound to a solid support, (d) optionally separating the BMC shell from the other constituents in the composition besides the BMC shell, (e) optionally washing the BMC shell-solid support complex with a solution that does not affect the binding between the first component and the second component such that further other constituents are separated from the BMC shell-solid support complex, (f) optionally repeating the step (d) washing, and (g) optionally separating the BMC shell from the solid support by dissociating the first component from the second component. In some embodiments, the BMC shell protein of the fusion protein is a BMC-P. In some embodiments, the BMC shell protein of the fusion protein is a BMC shell protein comprising a domain which forms the pentagonal vertice of the BMC shell.

The present invention also provides for a method for titrating a range of BMC shells with variable permeability, the method comprising: (a) optionally determining a desired permeability for a BMC shell, (b) forming a series of BMC shells with a variable amount of a fusion protein, comprising a BMC-P and a first component, relative to the amounts of the BMC-H and BMC-T shell proteins, (c) optionally purifying or isolating each BMC shell formed with a specific amount of the fusion protein relative the amounts of the BMC-H and BMC-T shell proteins using a second component bound to a solid support, (d) determining the permeability of each BMC shell formed with a specific amount of the fusion protein relative the amounts of the BMC-H and BMC-T shell proteins, and (e) optionally identifying the BMC shell of step (d) that has a permeability closest to the desired permeability determined in step (a). In some embodiments, the step (b) forming comprises using a method described herein, or in U.S. Pat. Nos. 11,130,788 and 12,071,455 (which are hereby incorporated by reference). In some embodiments, the step (b) forming comprises using an in vivo capped or ex vivo capped method.

Biotechnology, along with fundamental sciences, relies on luminescence produced from integrated luciferase enzymes for applications such as quantifying gene expression and describing protein biophysics. As biotechnological product complexity increases, systems which stably scaffold luciferase with precise nanoscale space/time organization appear necessary. Bacterial microcompartments (BMCs) self-assemble from a concert of outer shell proteins and interior cargos to form modular nanoscale containers used in biotechnology. It is a non-trivial project to load heterologous cargos into BMCs and uncertainties about shell molecular permeability may occur that alter cargo enzyme function. Disclosed herein is an approach which scaffolds luciferase inside a BMC. In some embodiments, the interior-localized enzyme to measure molecular permeability of the BMC shell. Comparing luciferase kinetics between a no-shell control, a shell with vertex gaps, and a sealed shell demonstrated a decrease in the permeability and overall kinetics between samples. In some embodiments, the protein shell compartmentalized luciferase (stable as monodisperse about 40 nm particles over a period, such as 1, 2, 3, or more months) may find utility in molecular diagnostics, nanofactory engineering, advanced materials, and fundamental scientific studies.

The present invention provides for a method for producing luminescent bacterial microcompartment (BMC) shells. In some embodiments, the method produces monodisperse about 40 nm protein particles with luminescent properties from the encapsulated firefly luciferase molecules and adjustable small-molecule permeability rates. In some embodiments, encapsulation of firefly luciferase is achieved in a precise and accurate manner by isopeptide formation with a “SpyTag” functionalized BMC shell protein in the BMC shell. In some embodiments, fusions of firefly luciferase to a reciprocal functionalizing domain (“SpyCatcher”) are screened for activity and it was found that a C-terminal fusion (“His-LucZ-SpyCatcher”) provide a high activity. In some embodiments, encapsulated luciferase is not only used to engineer a luminescent BMC shell, but also used to determine the small molecule permeability of ATP. When three types of samples are compared with stopped-flow spectrophotometry: a no shell, free floating His-LucZ-SpyCatcher, an uncapped BMC shell with encapsulated His-LucZ-SpyCatcher, and a capped BMC shell with encapsulated His-LucZ-SpyCatcher, mathematical modeling of the experimentally recorded kinetics of luminescence determined that the permeability to ATP increases nearly 50-fold in an uncapped compared to capped shell configuration. This method for producing luminescent BMC shells may find utility in further describing BMC shell molecular permeability, harnessing shells as molecular imaging agents, and as advanced light-producing materials.

In some embodiments, the system contains an unconjugated “SnoopTag” fusion to a BMC shell protein that is freely available for covalent targeting of additional functionality to the lumen of the BMC shell for application customization.

In some embodiments, the fusion protein and BMC shells of the present invention are expressed in cellular hosts or with cell-free technologies. In some embodiments, the fusion protein and BMC shells of the present invention are useful as molecular imaging agents, heterologous interfacing of shells within hydrogel or other biocompatible materials to create light-emitting biomaterials, and/or in-depth analysis of BMC shell pore structure and any subsequent changes to shell permeability.

In some embodiments, the fusion protein and BMC shells of the present invention are used to produce BMC shells trackable in cellular hosts via luminescence, or to engineer BMC shell protein pores for specific permeability rates to small-molecules. In some embodiments, the fusion protein and BMC shells of the present invention are used to produce luminescent BMC shells embeddable into hydrogels or other biocompatible materials which then creates light-producing materials. In some embodiments, the unconjugated “SnoopTag” functionalized BMC shell protein is used to specifically conjugate to the lumen on BMC shells additional application relevant cargo, such as an enzyme increasing reactive oxygen species (ROS) concentration that functions to target cells for destruction.

An advantage of the present invention is that the BMC shells have lower likelihood of interference from a system's immune system rather than being a viral-derived container. Also, BMC shells have advantages over viral-like particles because of their structural heterogeneity lending to opportunities for more specific functionalization to the proteins making the container. Further, BMC shells have lower likelihood of interference from a system's immune system being viral-derived containers.

Bacterial Microcompartments (BMCs) are selectively permeable proteinaceous organelles which encapsulate segments of metabolic pathways. A significant portion of global carbon fixation is mediated by these compartments in photosynthetic bacteria; other BMCs are involved in carbon catabolismand contribute to the fitness and virulence of some pathogenic bacteria. BMC architectures are beginning to be exploited by biotechnologists, however technical challenges including laborious purification protocols and inefficient targeting of cargo to the inside of the shells hinder engineering and characterization of these nanoparticles. These limitations are addressed through rational modifications to shell proteins which enable rapid purification of shells and precise encapsulation of cargo. These methods are applicable to functionally diverse shell systems, enabling their use in biotechnological and biomedical applications.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Design for evaluating ATP permeability of a model BMC shell. A.) BMC shells lacking pentamers contain about 5 nm vertex gaps (i.e. “uncapped” configuration). BMC shell structures were prepared in PyMol from PDB structure 5V74. BMC-H proteins are colored blue, BMC-T1 is colored dark green, BMC-T2 and BMC-T3 are colored light green, and BMC-P is colored yellow. B.) Zoom of area bounded in white in FIG. 1. C.) Fusion of a SpyTag/SnoopTag to BMC-T1 shell protein allows for covalent tethering of reciprocally-tagged-catcher cargos to the lumen of the BMC shell to evaluate the permeability of ATP to a BMC shell. D.) Transmission electron microscopy of purified uncapped LucZ-SpyCatcher BMC shells.

FIG. 2: Measuring ATP permeability of LucZ-SpyCatcher BMC shells with stopped-flow assays. A.) Stopped-flow assay comparison of uncapped versus capped shells versus a shell free LucZ in luciferin/MgCl₂ substrate (each trace represents five readings averaged to one). Concentration of shell samples in solution was 100 nM, while the No Shell sample was 1000 nM. Samples were incubated for one hour with or without 10-fold molar excess to shells in solution BMC-P capping protein in 1 mM luciferin/MgCl₂, 50 mM Tris-HCl, and 200 mM NaCl pH 8.0 buffer. Trends represented are from at least three independent experiments.

FIG. 3: Differential equation modeling of stop-flow enzyme kinetics to calculate permeability rate of capped versus uncapped shells. A.)-B.) Permeability coefficients are given by the rate of the limiting substrate (Px) and the rate of the intermediate complex (Py). Information regarding the equations, assumptions, and additional calculated values are found in the Supporting Information. A.) Capped shells B.) Uncapped shells.

FIG. S1: Purification of in vivo covalently loaded SpyCatcher-LucZ uncapped BMC shells. A.) MonoQ chromatogram of Uncapped BMC Shell with N-terminal SpyCatcher fusion to LucZ. B.) SDS-PAGE of 10 ug of SpyCatcher-LucZ Uncapped BMC Shells C.) DLS of a sample at 1 mg-mL in 50 mM Tris-HCl 200 mM NaCl, pH 8.0.

FIG. S2: Purification of in vivo covalently loaded LucZ-SpyCatcher uncapped BMC shells. A.) MonoQ chromatogram of Uncapped BMC Shell C-terminal SpyCatcher fusion to LucZ B.) SDS-PAGE of 10 ug of LucZ-SpyCatcher Uncapped BMC Shells C.) DLS of a sample at 1 mg-mL in 50 mM Tris-HCl 200 mM NaCl, pH 8.0.

FIG. S3: Luminescence activity comparison between LucZ and SpyCatcher fusion orientation. A.) Biochemical reactants and products for luciferase luminescence B.) Photons measured from 15 ug of BMC Full UnCapped Shells in 1 mM ATP and 1 mM Luciferin/MgCl₂ with 50 mM Tris-HCl, 200 mM NaCl, pH 8.0 buffer. Standard deviation represents an average of three technical replicates.

FIG. S4: Activity assay of His-LucZ-SpyCatcher loaded uncapped BMC shells. A.) Luminescence recorded in the presence of 1 mM luciferin and MgCl₂ substrate supplemented buffer with the final specified concentration of ATP and 100 ug of sample (100 nM final concentration for each protein sample). Standard deviation represents an average of two technical replicates.

FIG. S5: Capping pentamer protein purification, and work-flow for comparing shell permeability in uncapped versus capped configuration. A.) SDS-PAGE of two loaded amounts of BMC-P capping pentamer following StrepTrap purification and buffer exchange. B.) 5 mL samples for permeability experiments are prepared in a substrate buffer that lacks ATP and incubated overnight. After this, samples enzyme kinetics and structure are compared.

FIG. S6: LucZ-SpyCatcher Shells after incubation in substrate buffer. A.)-B.) DLS measurements of overnight incubated protein at 0.55 mg-mL in 1 mM luciferin and MgCl₂ substrate supplemented 50 mM Tris-HCl and 200 mM NaCl pH 8.0 buffer. A.) Uncapped B.) Capped C.)-F.) TEM negative stain of samples spotted on grids after incubation in substrate buffer. C.) and E.) Uncapped D.) and F.) Capped.

FIG. S7: Stopped-flow spectrophotometry of uncapped and capped BMC shells. A.) Stopped-flow schematic for shells (100 nM) in 1 mM luciferin and MgCl₂ substrate supplemented 50 mM Tris-HCl and 200 mM NaCl pH 8.0 buffer in one tube and 1 mM ATP in Tris-NaCl buffer in the other, and a 20% SDS-PAGE gel with 20 ug of uncapped or capped shells loaded following a 16-hour incubation B.) 0.5 second SX spectrophotometry kinetic measurement of five traces averaged together with four technical replicates each plotted. Inset is a SX biological replicate with two technical replicates plotted of uncapped versus capped shells C.) 0.5 second kinetic measurement average of three biological replicates containing five traces averaged together. Top inset contains a plot of C.), but with a 4 second kinetic measurement time period; 2nd inset is a plot with the Y-axis in Log 10 scale.

FIG. S8: Stopped-flow spectrophotometry of uncapped and capped shells in 10% and 20% PEG6000 buffer. A.) SX kinetic measurement of five traces averaged together after 16 hour incubation comparing samples in just Tris-NaCl substrate buffer or the indicated PEG6000 supplemented percentage in buffer. Inset is a plot of the full 10 second measurement B.) A 0.5 second SX kinetic measurement with only shell samples plotted for clarity.

FIG. S9: Stopped-flow spectrophotometry of MonoQ purified shells after capping. A.) Shells were incubated for 16-hours with 0, 0.2×, 2×, and 20× molar shells vertices: pentamer in luciferin/Mg substrate then purified with MonoQ anion exchange chromatography. Samples were then concentrated with a 100 kDa size exclusion amicon filter, incubated an additional 30 minutes with substrate, and then 50 nM shells was analyzed with stopped-flow spectrophotometry with ATP injected at 1 mM. All samples are statistically significant from each other (p value less than 0.005) and trend lines and standard deviation represent three technical replicates averaged with each measurement an average of five traces. B.) Same as A.) with the exception of using 70 nM shells and 0, 0.7×, and 7× shell vertices: pentamer capping ratios.

FIG. S10: Calculation of kM of uncapped and capped LucZ-SpyCatcher BMC shells. A.) Stopped-flow for 0.5 mg-mL protein in 1 mM luciferin and MgCl₂ substrate supplemented 50 mM Tris-HCl and 200 mM NaCl pH 8.0 buffer in one tube and indicated uM ATP in buffer in the other. B.) Result table of stretched exponential fitting C.) Plotting 1/kObs over 1/[ATP] to determine Km of samples D.) Table of calculated Km of uncapped and capped samples.

FIG. S11: Example fits for measurements taken without a shell or with a shell present. The inset is a zoomed in view of what happens in the first second, with gray zoom-boxes to indicate the relationship between the axes. Note that both cases have similar overall structures, with a quick, non-linear increase at short time, followed by a long decaying tail.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used in the specification and the appended claims, the singular forms “a”, “an”, and “the” include plural references unless the context clearly dictates otherwise. Thus, for example, reference to a “shell protein” includes a single shell protein molecule, and a plurality of shell protein molecules having the same, or similar, chemical formula, chemical and/or physical properties.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” refers to a value including 10%, or one, more than the stated value and 10%, or one, less than the stated value.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

These and other objects, advantages, and features of the invention will become apparent to those persons skilled in the art upon reading the details of the invention as more fully described below.

One aspect of the present invention provides for a method to encapsulate proteins or other materials that have been fused to short peptides within the shells of Bacterial Microcompartments (BMCs) using domain insertions and split bacterial adhesin technology. In parallel or alternatively this invention may also be used to decorate the exterior of the shells and hence this technology is a generalizable way to functionalize the inside and/or outside of BMCs. This is a fundamentally different encapsulation method compared to current practices in the field which depend on fusion of encapsulation peptides (EPs) to cargo. The method described here has been shown to be superior in terms of efficiency and modularity. Robust, programmable encapsulation of cargo is essential to realize the numerous potential applications of BMCs and BMC shell proteins. Therefore this technology elevates BMCs to the pantheon of protein nanoparticles (virus-like particles, ferritins, encapsulins etc.) that may be repurposed for diverse industrial and medical uses.

Certain protein sequences used herein are described in: U.S. Pat. No. 9,547,003 B2, and Zakeri, B., Fierer, J. O., Celik, E., Chittock, E. C., Schwarz-Linek, U., Moy, V. T., & Howarth, M. (2012). In some embodiments, the peptide tag forms a rapid covalent bond to a protein, through engineering a bacterial adhesin. Proceedings of the National Academy of Sciences of the United States of America, 109 (12), E690-7. Genes encoding these sequences are ordered as gene blocks through Integrated DNA Technologies. The fusion proteins and/or nucleic acids of the present invention are recombinant or constructed by human(s), or non-natural.

BMCs in general could have a number of applications in the biotech, pharmaceutical and medical industries. To realize many of these applications, efficient encapsulation of cargo as described in this invention is critical. Apart from nanoparticles, it is demonstrated that certain shell proteins can spontaneously form two-dimensional sheets. The integration of split bacterial adhesin domains into such proteins would allow for functionalization and/or crosslinking which may imbue these sheets with interesting material properties. Metabolic engineers can target heterologous pathways to subcellular compartments (e.g. mitochondria, peroxisomes, the periplasmic space) in vivo to increase product titers by reducing competing reactions, increasing precursor pool availability, decreasing toxicity of intermediates, faster enzyme kinetics etc. As the present invention allows efficient encapsulation of cargo, this enables metabolic engineers to reliably confine heterologous pathways to BMCs which may result in concomitant titer increases. A particular advantage of protein-based organelles like BMCs in comparison to lipid-based organelles is their more molecularly defined nature. This could enable more precise control over relative stoichiometries of encapsulated enzymes in multi-step pathways, which could further increase titers. The behavior of a catalyst is sensitive to its local environment and solution state. It is shown that enzymatic biocatalysis can be improved through scaffolding-based colocalization as well as through immobilization to inert carriers (e.g. solid supports or dispersed polymers) or via cross-linking. Therefore in industrial settings, catalyst stability and/or activity may be improved by encapsulation within BMCs, adhering to the outside of BMCs or arraying enzymes on the aforementioned protein sheets. Protein-based nanocontainers such as virus-like particles (VLPs), ferritins, encapsulins etc. have attracted interest in medicine as potential polyvalent antigen scaffolds, therapeutic-delivery vehicles and imaging contrast agents. BMCs could also be used in these applications and may offer advantages conferred by the selective permeability of their shells that allow more sophisticated layers of control. For example, the present invention could allow for efficient encapsulation of therapeutic agents that are activated by a small molecule (e.g. cancer biomarker) entering the shell and interacting with an encapsulated sensor. Another example is encapsulating contrast agents or other functional nanomaterials (e.g. upconverting nanoparticles). Localization to particular tissues may be possible because the outside of the shell can be functionalized with antibodies that recognize specific cell types. Encapsulation within a shell prevents the therapeutics/material from direct interaction with the host and therefore could be more stable or less likely to provoke immune responses.

As described herein, the present invention offers significant advantages over existing encapsulation technology in terms of efficiency and precision of encapsulation. In addition, because the number of SpyCatcher docking sites per particle is known and experiments have shown near quantitative binding by SpyTagged cargo, a much higher degree of control over number of cargo molecules encapsulated per particle is realized. The same attributes will apply to functionalization of the BMC shell exterior and shell-based protein layers. Additionally, the technology is modular insofar as the SpyCatcher domain can be engineered into different shell systems and the same SpyTagged protein/material constructs can be targeted to these new systems. This offers an advantage over EP-based encapsulation which requires changing the cognate EP on the cargo each time a new system is to be used.

In a particular embodiment, the present invention comprises the insertion of a split bacterial adhesin domain called “SpyCatcher” into a shell protein at a location facing the lumen of the shell and that does not disrupt the global folding of the protein or its ability to integrate into BMC shells. The SpyCatcher domain can also be appended to the N- or C-termini of shell proteins which project outwards from the shell in their normal context. Separately, proteins or inorganic materials that are to be encapsulated or adhered (referred to hereafter as “cargo”) can be fused to a short (about 13 residue) amino acid sequence called “SpyTag.” When co-present either in vivo or in vitro, the SpyCatcher-shell protein and SpyTag-cargo associate and form an isopeptide bond that covalently links the two polypeptides. For SpyCatcher insertions, the topology of the shell proteins guarantees that SpyTag-cargo are localized within the lumen of the BMC shells and are tethered in place via the isopeptide bond. In contrast, if SpyCatcher is fused to the N- or C-termini of shell proteins, the SpyTagged constructs would localize to the outside of a shell. An inverted incarnation of the method wherein SpyTag is fused to shell proteins and SpyCatcher is fused to cargo has also been demonstrated. Likewise Spytag can be fused to externally projecting shell protein termini providing a means for capture of shells using, for example, a Spycatcher functionalized matrix.

This is a fundamentally different encapsulation/functionalization method in comparison to current practices in the field which depend on fusion of so-called “encapsulation peptides” (EPs) to cargo. EPs typically consist of short (about 17 residues) polypeptide sequences that form amphipathic alpha-helices and which interact non-covalently with the inner surface of BMC shells through non-covalent hydrophobic and/or electrostatic interactions. Each different shell system (i.e. from different organisms or different biochemical subtypes) has its own EP(s) and usually undefined cognate docking site(s) on the lumenal surface of the shell. Numerous studies have shown that targeting heterologous cargo via EP fusions is rarely efficient-such heterologous cargo comprises a very minor amount of total protein in these shell preparations. In contrast, covalent association of SpyTag-cargo with SpyCatcher-shells (or the inverse) in theory offers a 1:1 stoichiometry between shell protein and cargo and therefore enables high efficiency of encapsulation and the creation of molecularly defined BMC cores/outside surfaces. The usage of split bacterial adhesin domains for bacterial microcompartment-associated applications is novel and allows cargo to be generated independent of the shell protein (e.g. proteins expressed in a different host or use of abiotic materials).

The fundamental challenge for development of the SpyCatcher insertional fusions is identification of an architecture for the SpyCatcher domain that satisfies at least one, two or three of the following criteria:
1. Insertion at a location that is solvent exposed to the lumen, or alternatively, the exterior of BMC shells.
2. Insertion at a location that does not disrupt proper folding of the shell protein which would preclude proper integration into the greater shell structure.
3. The SpyCatcher domain is positioned such that SpyTag-cargo (or the inverse) has the steric freedom to associate and form covalent linkages.

A putative site satisfying these criteria is identified in a particular shell protein from the HO shell system found in the bacterium Haliangium ochraceum (locus tag: 5812) by a combination of structural analysis of the protein's solved crystal structure and sequence analysis of protein orthologs. The chosen site is a short, poorly conserved loop consisting of small, flexible amino acids of the sequence “AGSGA” (SEQ ID NO:6) which is located in the lumenal side of an assembled shell. Domain insertion is commonly done at poorly-conserved, flexible loops due the reduced chance of disrupting the target protein function in comparison to insertion within conserved, structured regions of proteins. Finally, the HO-5812 protein is homotrimeric and the insertion sites in a fully formed trimer are approximately 38.7 Angstrom apart which is speculated to be large enough to afford cargo sufficient steric freedom to associate with the SpyCatcher-fused shell protein. Because this region is shared amongst HO-5812 homologs in other shell systems (e.g. Propanediol Utilization [Pdu] and Ethanolamine Utilization [Eut] BMCs), it is believed that the methodology is amenable to application in other such shell systems.

After creation of the fusion constructs using routine molecular biology methods, tests showed unambiguously that the HO-5812-SpyCatcher and HO-5812-SpyTag fusion proteins are capable of proper integration into HO shells. Furthermore, when a respective SpyTag or SpyCatcher fused fluorescent protein is co-expressed with the shell proteins, a size-shift is observed by SDS-PAGE analysis and shell preparations are fluorescent. These two lines of evidence validate the method.

Methods of making and using BMCs, including in vitro assembly and HO shells, are taught in U.S. Patent Provisional Application Ser. Nos. 62/509,553 and 62/671,369; U.S. Patent Application Publication Nos. 2012/0210459, 2013/0133102, 2015/0026840, 2016/0222068, 2017/0107523, 2018/0057546; U.S. Pat. Nos. 10,913,777; 11,130,788; 11,198,880; and, 12,071,455; and, PCT International Patent Application Nos. WO 2011/017458 and WO 2011/094765; which are all incorporated by reference in their entireties.

### CITED REFERENCES

(1) Kerfeld, C. A.; Aussignargues, C.; Zarzycki, J.; Cai, F.; Sutter, M. Bacterial Microcompartments. Nat. Rev. Microbiol. 2018, 16 (5), 277-290.
(2) Sutter, M.; Melnicki, M. R.; Schulz, F.; Woyke, T.; Kerfeld, C. A. A Catalog of the Diversity and Ubiquity of Bacterial Microcompartments. Nat. Commun. 2021, 12 (1), 3809.
(3) Steele, J. F. C.; Kerfeld, C. A. Encyclopedia of Biological Chemistry III. 2021, 108-122.
(4) Sutter, M.; Greber, B.; Aussignargues, C.; Kerfeld, C. A. Assembly Principles and Structure of a 6.5-MDa Bacterial Microcompartment Shell. Science 2017, 356 (6344), 1293-1297.
(5) Kerfeld, C. A.; Sawaya, M. R.; Tanaka, S.; Nguyen, C. V.; Phillips, M.; Beeby, M.; Yeates, T. O. Protein Structures Forming the Shell of Primitive Bacterial Organelles. Science 2005, 309 (5736), 936-938.
(6) Klein, M. G.; Zwart, P.; Bagby, S. C.; Cai, F.; Chisholm, S. W.; Heinhorst, S.; Cannon, G. C.; Kerfeld, C. A. Identification and Structural Analysis of a Novel Carboxysome Shell Protein with Implications for Metabolite Transport. J. Mol. Biol. 2009, 392 (2), 319-333.
(7) Ferlez, B. H.; Kirst, H.; Greber, B. J.; Nogales, E.; Sutter, M.; Kerfeld, C. A. Heterologous Assembly of Pleomorphic Bacterial Microcompartment Shell Architectures Spanning the Nano-to Microscale. Adv. Mater. 2023, 35 (23), e2212065.
(8) Tan, Y. Q.; Ali, S.; Xue, B.; Teo, W. Z.; Ling, L. H.; Go, M. K.; Lv, H.; Robinson, R. C.; Narita, A.; Yew, W. S. Structure of a Minimal A-Carboxysome-Derived Shell and Its Utility in Enzyme Stabilization. Biomacromolecules 2021, 22 (10), 4095-4109.
(9) Cesle, E. E.; Filimonenko, A.; Tars, K.; Kalnins, G. Variety of Size and Form of GRM2 Bacterial Microcompartment Particles. Protein Sci. 2021, 30 (5), 1035-1043.
(10) Sutter, M.; Laughlin, T. G.; Sloan, N. B.; Serwas, D.; Davies, K. M.; Kerfeld, C. A. Structure of a Synthetic β-Carboxysome Shell. Plant Physiol. 2019, 181 (3), 1050-1058.
(11) Evans, S. L.; Al-Hazeem, M. M. J.; Mann, D.; Smetacek, N.; Beavil, A. J.; Sun, Y.; Chen, T.; Dykes, G. F.; Liu, L.-N.; Bergeron, J. R. C. Single-Particle Cryo-EM Analysis of the Shell Architecture and Internal Organization of an Intact α-Carboxysome. Structure 2023, 31 (6), 677-688.
(12) Greber, B. J.; Sutter, M.; Kerfeld, C. A. The Plasticity of Molecular Interactions Governs Bacterial Microcompartment Shell Assembly. Structure 2019, 27 (5), 749-763.
(13) Zhou, R.-Q.; Jiang, Y.-L.; Li, H.; Hou, P.; Kong, W.-W.; Deng, J.-X.; Chen, Y.; Zhou, C.-Z.; Zeng, Q. Structure and Assembly of the α-Carboxysome in the Marine Cyanobacterium Prochlorococcus. Nat. Plants 2024, 1-12.
(14) Ni, T.; Jiang, Q.; Ng, P. C.; Shen, J.; Dou, H.; Zhu, Y.; Radecke, J.; Dykes, G. F.; Huang, F.; Liu, L.-N.; Zhang, P. Intrinsically Disordered CsoS2 Acts as a General Molecular Thread for α-Carboxysome Shell Assembly. Nat. Commun. 2023, 14 (1), 5512.
(15) Tanaka, S.; Kerfeld, C. A.; Sawaya, M. R.; Cai, F.; Heinhorst, S.; Cannon, G. C.; Yeates, T.
O. Atomic-Level Models of the Bacterial Carboxysome Shell. Science 2008, 319 (5866), 1083-1086.
(16) Kerfeld, C. A.; Erbilgin, O. Bacterial Microcompartments and the Modular Construction of Microbial Metabolism. Trends Microbiol. 2014, 23 (1), 22-34.
(17) Liu, L.-N. Advances in the Bacterial Organelles for CO2 Fixation. Trends Microbiol. 2022, 30 (6), 567-580.
(18) Turmo, A.; Gonzalez-Esquer, C. R.; Kerfeld, C. A. Carboxysomes: Metabolic Modules for CO2 Fixation. FEMS Microbiol. Lett. 2017, 364 (18), 1000118.
(19) Faulkner, M.; Szabó, I.; Weetman, S. L.; Sicard, F.; Huber, R. G.; Bond, P. J.; Rosta, E.; Liu, L.-N. Molecular Simulations Unravel the Molecular Principles That Mediate Selective Permeability of Carboxysome Shell Protein. Sci. Rep. 2020, 10 (1), 17501.
(20) Tsai, Y.; Sawaya, M. R.; Cannon, G. C.; Cai, F.; Williams, E. B.; Heinhorst, S.; Kerfeld, C.
A.; Yeates, T. O. Structural Analysis of CsoSIA and the Protein Shell of the Halothiobacillus Neapolitanus Carboxysome. PLoS Biol. 2007, 5 (6), e144.
(21) Mahinthichaichan, P.; Morris, D. M.; Wang, Y.; Jensen, G. J.; Tajkhorshid, E. Selective Permeability of Carboxysome Shell Pores to Anionic Molecules. J. Phys. Chem. B 2018, 122 (39), 9110-9118.
(22) Trettel, D. S.; Neale, C.; Zhao, M.; Gnanakaran, S.; Gonzalez-Esquer, C. R. Monatomic Ions Influence Substrate Permeation across Bacterial Microcompartment Shells. Sci. Rep. 2023, 13 (1), 15738.
(23) Park, J.; Chun, S.; Bobik, T. A.; Houk, K. N.; Yeates, T. O. Molecular Dynamics Simulations of Selective Metabolite Transport across the Propanediol Bacterial Microcompartment Shell. J. Phys. Chem. B 2017, 121 (34), 8149-8154.
(24) Sarkar, D.; Maffeo, C.; Sutter, M.; Aksimentiev, A.; Kerfeld, C.; Vermaas, J. Atomic View of Photosynthetic Metabolite Permeability Pathways and Confinement in Cyanobacterial Carboxysomes. 2024.
(25) Penrod, J. T.; Roth, J. R. Conserving a Volatile Metabolite: A Role for Carboxysome-Like Organelles in Salmonella Enterica. J. Bacteriol. 2006, 188 (8), 2865-2874.
(26) Havemann, G. D.; Sampson, E. M.; Bobik, T. A. PduA Is a Shell Protein of Polyhedral Organelles Involved in Coenzyme B 12-Dependent Degradation of 1,2-Propanediol in Salmonella Enterica Serovar Typhimurium LT2. J. Bacteriol. 2002, 184 (5), 1253-1261.
(27) Chowdhury, C.; Chun, S.; Pang, A.; Sawaya, M. R.; Sinha, S.; Yeates, T. O.; Bobik, T. A. Selective Molecular Transport through the Protein Shell of a Bacterial Microcompartment Organelle. Proc. Natl. Acad. Sci. 2015, 112 (10), 2990-2995.
(28) Chowdhury, C.; Chun, S.; Sawaya, M. R.; Yeates, T. O.; Bobik, T. A. The Function of the PduJ Microcompartment Shell Protein Is Determined by the Genomic Position of Its Encoding Gene. Mol. Microbiol. 2016, 101 (5), 770-783.
(29) Chowdhury, C.; Bobik, T. A. Engineering the PduT Shell Protein to Modify the Permeability of the 1,2-Propanediol Microcompartment of Salmonella. Microbiology 2019, 165 (12), 1355-1364.
(30) Cai, F.; Menon, B. B.; Cannon, G. C.; Curry, K. J.; Shively, J. M.; Heinhorst, S. The Pentameric Vertex Proteins Are Necessary for the Icosahedral Carboxysome Shell to Function as a CO2 Leakage Barrier. PLoS ONE 2009, 4 (10), e7521.
(31) Lee, M. F. S.; Jakobson, C. M.; Tullman-Ercek, D. Evidence for Improved Encapsulated Pathway Behavior in a Bacterial Microcompartment through Shell Protein Engineering. ACS Synth. Biol. 2017, 6 (10), 1880-1891.
(32) Huang, J.; Jiang, Q.; Yang, M.; Dykes, G. F.; Weetman, S. L.; Xin, W.; He, H.-L.; Liu, L.-N. Probing the Internal PH and Permeability of a Carboxysome Shell. Biomacromolecules 2022, 23 (10), 4339-4348.
(33) Rae, B. D.; Long, B. M.; Badger, M. R.; Price, G. D. Structural Determinants of the Outer Shell of β-Carboxysomes in Synechococcus Elongatus PCC 7942: Roles for CcmK2, K3-K4, CcmO, and CcmL. PLoS ONE 2012, 7 (8), e43871.
(34) Cai, F.; Sutter, M.; Bernstein, S. L.; Kinney, J. N.; Kerfeld, C. A. Engineering Bacterial Microcompartment Shells: Chimeric Shell Proteins and Chimeric Carboxysome Shells. ACS Synth. Biol. 2015, 4 (4), 444-453.
(35) Cameron, J. C.; Wilson, S. C.; Bernstein, S. L.; Kerfeld, C. A. Biogenesis of a Bacterial Organelle: The Carboxysome Assembly Pathway. Cell 2013, 155 (5), 1131-1140.
(36) Yang, M.; Wenner, N.; Dykes, G. F.; Li, Y.; Zhu, X.; Sun, Y.; Huang, F.; Hinton, J. C. D.; Liu, L.-N. Biogenesis of a Bacterial Metabolosome for Propanediol Utilization. Nat. Commun. 2022, 13 (1), 2920.
(37) Huseby, D. L.; Roth, J. R. Evidence That a Metabolic Microcompartment Contains and Recycles Private Cofactor Pools. J. Bacteriol. 2013, 195 (12), 2864-2879.
(38) Sommer, M.; Sutter, M.; Gupta, S.; Kirst, H.; Turmo, A.; Lechno-Yossef, S.; Burton, R. L.; Saechao, C.; Sloan, N. B.; Cheng, X.; Chan, L.-J. G.; Petzold, C. J.; Fuentes-Cabrera, M.; Ralston, C. Y.; Kerfeld, C. A. Heterohexamers Formed by CcmK3 and CcmK4 Increase the Complexity of Beta Carboxysome Shells. Plant Physiol. 2019, 179 (1), 156-167.
(40) Sun, Y.; Wollman, A. J. M.; Huang, F.; Leake, M. C.; Liu, L.-N. Single-Organelle Quantification Reveals Stoichiometric and Structural Variability of Carboxysomes Dependent on the Environment. Plant Cell 2019, 31 (7), 1648-1664.
(41) Sommer, M.; Cai, F.; Melnicki, M.; Kerfeld, C. A. β-Carboxysome Bioinformatics: Identification and Evolution of New Bacterial Microcompartment Protein Gene Classes and Core Locus Constraints. J. Exp. Bot. 2017, 68 (14), 3841-3855.
(42) Hagen, A.; Sutter, M.; Sloan, N.; Kerfeld, C. A. Programmed Loading and Rapid Purification of Engineered Bacterial Microcompartment Shells. Nat. Commun. 2018, 9 (1), 2881.
(43) Trettel, D. S.; Resager, W.; Ueberheide, B. M.; Jenkins, C. C.; Winkler, W. C. Chemical Probing Provides Insight into the Native Assembly State of a Bacterial Microcompartment. Structure 2022, 30 (4), 537-550.e5.
(44) Jiang, Q.; Li, T.; Yang, J.; Aitchison, C. M.; Huang, J.; Chen, Y.; Huang, F.; Wang, Q.; Cooper, A. I.; Liu, L.-N. Synthetic Engineering of a New Biocatalyst Encapsulating [NiFe]-Hydrogenases for Enhanced Hydrogen Production. J. Mater. Chem. B 2023, 11 (12), 2684-2692.
(45) Nguyen, N. D.; Pulsford, S. B.; Hee, W. Y.; Rae, B. D.; Rourke, L. M.; Price, G. D.; Long, B. M. Towards Engineering a Hybrid Carboxysome. Photosynth. Res. 2023, 156 (2), 265-277.
(46) Kirst, H.; Ferlez, B. H.; Lindner, S. N.; Cotton, C. A. R.; Bar-Even, A.; Kerfeld, C. A. Toward a Glycyl Radical Enzyme Containing Synthetic Bacterial Microcompartment to Produce Pyruvate from Formate and Acetate. Proc. Natl. Acad. Sci. 2022, 119 (8), e2116871119.
(47) Li, T.; Jiang, Q.; Huang, J.; Aitchison, C. M.; Huang, F.; Yang, M.; Dykes, G. F.; He, H.-L.; Wang, Q.; Sprick, R. S.; Cooper, A. I.; Liu, L.-N. Reprogramming Bacterial Protein Organelles as a Nanoreactor for Hydrogen Production. Nat. Commun. 2020, 11 (1), 5448.
(48) Wagner, H. J.; Capitain, C. C.; Richter, K.; Nessling, M.; Mampel, J. Engineering Bacterial Microcompartments with Heterologous Enzyme Cargos. Eng. Life Sci. 2016, 17 (1), 36-46.
(49) Lawrence, A. D.; Frank, S.; Newnham, S.; Lee, M. J.; Brown, I. R.; Xue, W.-F.; Rowe, M. L.; Mulvihill, D. P.; Prentice, M. B.; Howard, M. J.; Warren, M. J. Solution Structure of a Bacterial Microcompartment Targeting Peptide and Its Application in the Construction of an Ethanol Bioreactor. ACS Synth. Biol. 2014, 3 (7), 454-465.
(50) Choudhary, S.; Quin, M. B.; Sanders, M. A.; Johnson, E. T.; Schmidt-Dannert, C. Engineered Protein Nano-Compartments for Targeted Enzyme Localization. PLoS ONE 2012, 7 (3), e33342.
(51) Li, T.; Chang, P.; Chen, W.; Shi, Z.; Xue, C.; Dykes, G. F.; Huang, F.; Wang, Q.; Liu, L.-N. Nanoengineering Carboxysome Shells for Protein Cages with Programmable Cargo Targeting. ACS Nano 2024.
(52) Lassila, J. K.; Bernstein, S. L.; Kinney, J. N.; Axen, S. D.; Kerfeld, C. A. Assembly of Robust Bacterial Microcompartment Shells Using Building Blocks from an Organelle of Unknown Function. J. Mol. Biol. 2014, 426 (11), 2217-2228.
(53) Ferlez, B.; Sutter, M.; Kerfeld, C. A. A Designed Bacterial Microcompartment Shell with Tunable Composition and Precision Cargo Loading. Metab. Eng. 2019, 54, 286-291.
(54) Dale, R.; Ohmuro-Matsuyama, Y.; Ueda, H.; Kato, N. Mathematical Model of the Firefly Luciferase Complementation Assay Reveals a Non-Linear Relationship between the Detected Luminescence and the Affinity of the Protein Pair Being Analyzed. PLoS ONE 2016, 11 (2), e0148256.
(55) Dale, R.; Ohmuro-Matsuyama, Y.; Ueda, H.; Kato, N. Non-Steady State Analysis of Enzyme Kinetics in Real Time Elucidates Substrate Association and Dissociation Rates: Demonstration with Analysis of Firefly Luciferase Mutants. Biochemistry 2019, 58 (23), 2695-2702.
(56) Zakeri, B.; Fierer, J. O.; Celik, E.; Chittock, E. C.; Schwarz-Linek, U.; Moy, V. T.; Howarth, M. Peptide Tag Forming a Rapid Covalent Bond to a Protein, through Engineering a Bacterial Adhesin. Proc. Natl. Acad. Sci. 2012, 109 (12), E690-E697.
(57) Ribeiro, C.; Silva, J. C. G. E. da. Kinetics of Inhibition of Firefly Luciferase by Oxyluciferin and Dehydroluciferyl-Adenylate. Photochem. Photobiol. Sci. 2008, 7 (9), 1085-1090.
(58) Yang, N. J.; Hinner, M. J. Site-Specific Protein Labeling, Methods and Protocols. Methods Mol. Biol. 2014, 1266, 29-53.
(59) Vermaas, J. V.; Dixon, R. A.; Chen, F.; Mansfield, S. D.; Boerjan, W.; Ralph, J.; Crowley, M. F.; Beckham, G. T. Passive Membrane Transport of Lignin-Related Compounds. Proc. Natl. Acad. Sci. 2019, 116 (46), 23117-23123.
(60) Melnicki, M. R.; Sutter, M.; Kerfeld, C. A. Evolutionary Relationships among Shell Proteins of Carboxysomes and Metabolosomes. Curr. Opin. Microbiol. 2021, 63, 1-9.
(61) Du, P.; Xu, S.; Xu, Z.; Wang, Z. Bioinspired Self-Assembling Materials for Modulating Enzyme Functions. Adv. Funct. Mater. 2021, 31 (38), 2104819.
(62) Küchler, A.; Yoshimoto, M.; Luginbühl, S.; Mavelli, F.; Walde, P. Enzymatic Reactions in Confined Environments. Nat. Nanotechnol. 2016, 11 (5), 409-420.
(63) Caparco, A. A.; Dautel, D. R.; Champion, J. A. Protein Mediated Enzyme Immobilization. Small 2022, 18 (19), e2106425.
(64) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J.-Y.; White, D. J.; Hartenstein, V.; Eliceiri, K.; Tomancak, P.; Cardona, A. Fiji: An Open-Source Platform for Biological-Image Analysis. Nat. Methods 2012, 9 (7), 676-682.
(65) Mirdita, M.; Schütze, K.; Moriwaki, Y.; Heo, L.; Ovchinnikov, S.; Steinegger, M. ColabFold: Making Protein Folding Accessible to All. Nat. Methods 2022, 19 (6), 679-682.
(66) Virtanen, P.; Gommers, R.; Oliphant, T. E.; Haberland, M.; Reddy, T.; Cournapeau, D.; Burovski, E.; Peterson, P.; Weckesser, W.; Bright, J.; Walt, S. J. van der; Brett, M.; Wilson, J.; Millman, K. J.; Mayorov, N.; Nelson, A. R. J.; Jones, E.; Kern, R.; Larson, E.; Carey, C. J.; Polat, i.; Feng, Y.; Moore, E. W.; VanderPlas, J.; Laxalde, D.; Perktold, J.; Cimrman, R.; Henriksen, I.; Quintero, E. A.; Harris, C. R.; Archibald, A. M.; Ribeiro, A. H.; Pedregosa, F.; Mulbregt, P. van; Contributors, S. 1 0; Vijaykumar, A.; Bardelli, A. P.; Rothberg, A.; Hilboll, A.; Kloeckner, A.; Scopatz, A.; Lee, A.; Rokem, A.; Woods, C. N.; Fulton, C.; Masson, C.; Häggström, C.; Fitzgerald, C.; Nicholson, D. A.; Hagen, D. R.; Pasechnik, D. V.; Olivetti, E.; Martin, E.; Wieser, E.; Silva, F.; Lenders, F.; Wilhelm, F.; Young, G.; Price, G. A.; Ingold, G.-L.; Allen, G. E.; Lee, G. R.; Audren, H.; Probst, I.; Dietrich, J. P.; Silterra, J.; Webber, J. T.; Slavič, J.; Nothman, J.; Buchner, J.; Kulick, J.; Schönberger, J. L.; Cardoso, J. V. de M.; Reimer, J.; Harrington, J.; Rodríguez, J. L. C.; Nunez-Iglesias, J.; Kuczynski, J.; Tritz, K.; Thoma, M.; Newville, M.; Kümmerer, M.; Bolingbroke, M.; Tartre, M.; Pak, M.; Smith, N. J.; Nowaczyk, N.; Shebanov, N.; Pavlyk, O.; Brodtkorb, P. A.; Lee, P.; McGibbon, R. T.; Feldbauer, R.; Lewis, S.; Tygier, S.; Sievert, S.; Vigna, S.; Peterson, S.; More, S.; Pudlik, T.; Oshima, T.; Pingel, T. J.; Robitaille, T. P.; Spura, T.; Jones, T. R.; Cera, T.; Leslie, T.; Zito, T.; Krauss, T.; Upadhyay, U.; Halchenko, Y. O.; Vázquez-Baeza, Y. SciPy 1.0: Fundamental Algorithms for Scientific Computing in Python. Nat. Methods 2020, 17 (3), 261-272.
(67) Marquardt, D. W. An Algorithm for Least-Squares Estimation of Nonlinear Parameters. J. Soc. Ind. Appl. Math. 1963, 11 (2), 431-441.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Quantitative Measurement of Molecular Permeability to a Synthetic Bacterial Microcompartment Shell System

Naturally evolved and synthetically designed forms of compartmentalization benefit encapsulated function by increasing local concentrations of substrates and/or protecting cargo from destabilizing environments and inhibitors. Crucial to understanding the fundamental principles of compartmentalization are experimental systems enabling the measurement of permeability rates of small-molecules. Here, we report the experimental measurement of the small-molecule permeability of a 40 nm icosahedral bacterial microcompartment shell. This was accomplished by heterologous loading of light-producing luciferase enzymes and kinetic measurement of luminescence using stopped-flow spectrophotometry. Compared to free enzyme, luminescence signal kinetics were slower when the luciferase was encapsulated into bacterial microcompartment shells. The results indicate substrates and products still can exchange across the shell and modeling of the experimental data suggest that a 50× permeability rate increase occurs when shell vertex positions were vacant. Overall, our results suggest design considerations for the construction of heterologous bacterial microcompartment shells systems and compartmentalized function at the nanoscale.

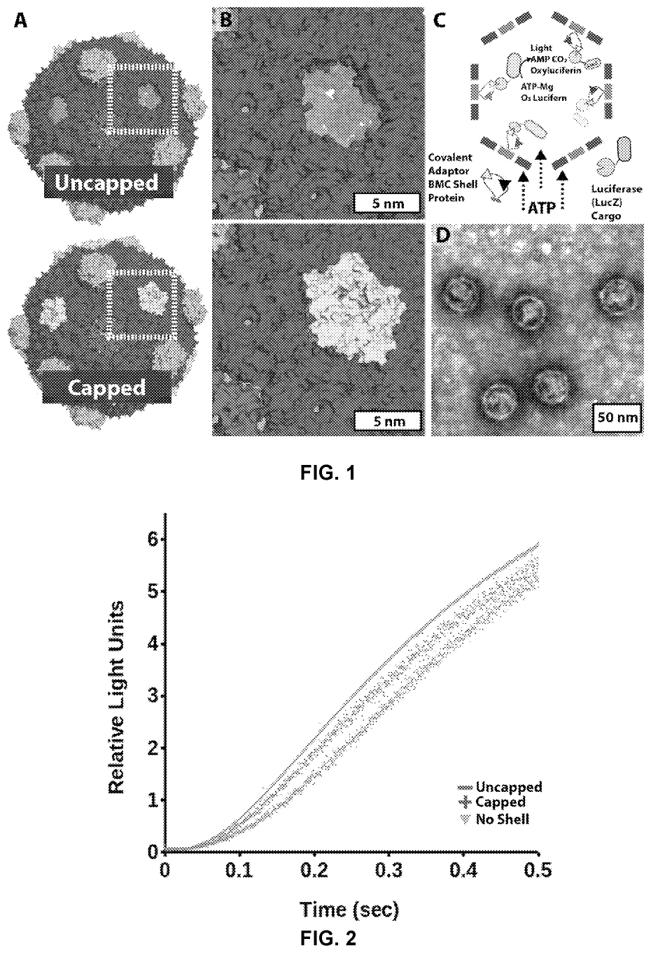

Accordingly, we designed a strategy in which lumen-localized cargo generates an output directly coupled to the molecular permeability of the shell. Numerous researchers have successfully incorporated active non-native enzymatic cargos into a BMC shell lumen^{8,44-51}. Here, we heterologously load a programmable BMC shell^{4,42,52,53} with firefly luciferase to act as a permeability reporter for the small-molecule adenosine triphosphate (ATP). The system relies on the in vivo loading of this versatile, well characterized luminescent cargo^{54,55} into a shell's lumen with the SpyTag/SpyCatcher split-adhesion covalent targeting system^{42,56} Luminescence producing shells, lacking vertices were purified in and compared to counterpart shell samples with all vertices occupied (FIG. 1). Stopped-flow spectrophotometry of the kinetics of light-production measured slower rising of luminescence signal in shells in the capped versus uncapped configuration. Differential equation modeling of the experimental data revealed that capped shells are about 50× less permeable. Notably, these data support that polar small-molecules exchange with a shell's lumen. Our approach is broadly applicable to other shell systems where it can be used to characterize engineered BMC shells and more broadly, to uncover fundamental design principles of compartmentalization at the nanoscale.

### RESULTS

### Characterization of Firefly Luciferase Loaded BMC Shells

A previously published Haliangium ochraceum synthetic uncapped BMC shell with a BMC-T1 SpyTag001/SnoopTag loop insertion was selected as our starting BMC shell chassis to engineer a reporter enzyme system for ATP shell permeability (FIG. 1)⁴⁶. The StrepII-tagged BMC-H shell protein in the synthetic BMC shell protein operon enables purifying shells with shell BMC-P vertex vacancies (i.e. an uncapped configuration). The size of the vertex gaps left by where the BMC-P protein inserts is about 5 nm in diameter. Vacancies at the vertices can be capped by adding exogenous purified BMC-P to the solution^{4,42}. The type and number of oligomer shell proteins in this shell are: 60 hexamers as the facets, 20 trimers located at the center of each facet, and 12 pentamers at the vertices⁴. In vivo co-expression of this operon with reciprocally tagged “catcher” cargos inside a cell can load BMC shells with covalently bound cargo to the shell's lumen⁴⁶. We co-expressed this BMC shell operon in vivo with alternative N- and C-terminal SpyCatcher fusions to Nipponoluciola cruciata firefly luciferase (LucZ), which has been developed into a versatile bioluminescent reporter system (FIG. S1-3)^{54,55}. Following shell purification and anion exchange polishing to remove broken shell impurities, both fusion orientations contained about 40 nm shell-sized particles measured by dynamic light scattering (DLS) (FIG. S1-2). However, the LucZ-SpyCatcher C-terminal fusion orientation was approximately two-fold higher in total luminescence (FIG. S3). An activity assay of 100 ug of BMC shells loaded with LucZ-SpyCatcher detected as low as 0.5 nM of ATP in solution under our assay conditions (FIG. S4).

### Measuring ATP Permeability Through a BMC Shell

Luminescence produced from the covalently bound lumen-located LucZ-SpyCatcher serves a reporter for permeability of the shell to ATP. In this system, LucZ is confined to the BMC lumen by the covalent tether and is not anticipated to exchange with the bulk outside the shell lumen. LucZ uses ATP, luciferin, and oxygen to generate visible light, so luminescence activity supports substrates crossing the BMC shell and therefore any change in the kinetics of luminescence implies an altered permeability rate. LucZ-containing shell samples had about 100 nM final concentration of protein in solution, while purified BMC-P was added to a second sample at approximately 10-molar excess of shells in solution (FIG. S5). At this ratio, it is anticipated that shell's vertices are fully capped and do not permit passage of molecules larger than the shell's pores⁴². After incubation in a luciferin and MgCl₂-containing buffer at room temperature for at least 16 hours, DLS, transmission electron microscopy (TEM) negative stain, and an luminescence assay in the presence of ATP supported the preservation of shell size, structure, and luciferase activity (FIG. S6).

Reaction kinetics were then measured with stopped-flow spectrophotometry (FIG. 2, FIG. S7-9). BMC shell samples pre-incubated in the substrate buffer were placed in one injection syringe, while a solution of ATP in the buffer was placed in the other injection syringe. Upon mixing, a rise in luminescence in both capped and uncapped shells occurred, which peaked after approximately 1 second. Notably, the uncapped shell samples' luminescence signal intensity rose with a steeper slope than the capped shell sample reproducibly. We then affinity purified a “No Shell” His-LucZ-SpyCatcher control and compared the kinetics of luminescence production with His-LucZ-SpyCatcher covalently tethered inside via SpyTag in uncapped or capped shells (FIG. 2, FIG. S7C). When the No Shell control in the substrate buffer is injected, this sample has faster kinetics than both the uncapped and capped samples. We hypothesized that adding a molecular crowder would slow luminescence kinetics due to an increase in solution viscosity decreasing Brownian diffusion rates of molecules in the system. This occurred and with similarity to previously reported trends: luminescence rose fastest in the shell-lacking control followed by uncapped and then capped samples (FIG. S8). An exception is the No Shell control in 20% PEG, which appears to slow unexpectedly more than the 10% PEG and buffer-only samples. We note that this sample routinely had visible to the eye protein-like aggregation, hinting that enzymes without a shell were not as stable under the 20% PEG6000 condition resulting in the altered trend in kinetics. We also tested the influence of varying the concentration of capping pentamer in solution (FIG. S9). To avoid the influence of viscosity during measurements due to excess pentamer in solution, we capped and then removed free pentamer by incubating wiffle shells with pentamer overnight in Luciferin/MgCl2 substrate buffer and then anion-exchange purified samples followed by centrifugation with 100 kDa cutoff Amicon filter/buffer exchange, which retains shells, while free pentamer collects in the filtrate. Overall, as observed in FIG. S9, shells lacking a capping pentamer have the fastest kinetics, while shells with a 1:10 sub-stoichiometric amount of pentamer have intermediate kinetics between an uncapped versus full capped system and samples with excess pentamer have the slowest kinetics.

To determine the overall effect of encapsulation on enzymatic activity we calculated the Km* for ATP. We term this Km* to avoid confusion with the Km intrinsic to luciferase in a freely diffusing system, which is approximately 15 μM⁵⁷. We found that Km* ATP for capped samples was approximately 8-fold higher (about 16 μM ATP for uncapped versus about 119 μM ATP for capped) (FIG. S10). This further strengthens the notion that BMC shells create a diffusion barrier to ATP, but are not fully impermeable to it.

As shown in FIG. 2, it is clear that the uncapped BMC shells are more permeable than the capped shells because of the increase of LucZ's kinetic rate in the uncapped samples. Intuitively, the absence of the 12 pentameric vertices of the BMC icosahedron provides an opening through which small molecules can permeate more freely, whereas small molecules that encounter capped shells have a higher probability of being reflected back into solution rather than entering the shell. Turning this qualitative observation into a quantitative model depends on selecting a model itself. Because the general luminescence pattern is similar for enzymes in bulk solution and samples containing enzymes tethered within a shell (with an initial non-linear rise followed by a peak and a nonlinear decline in luminance) multiple models were tested. The model that fits best under all conditions is one where intermediate products inhibit enzyme activity in a two-step reaction^{54,55}. The selected model also agrees best with the established luciferase reaction mechanism of ATP first reacting with D-luciferin to D-luciferyl-AMP and PPi; D-luciferyl-AMP is then converted to oxyluciferin and AMP upon O₂ reacting releasing light and CO₂⁵⁷. The formal differential equations solved numerically are derived in the Supporting Information. The differential equations can be fit either for an individual trace, or instead, use a shared set of thermodynamic and kinetic parameters with varying initial concentrations.

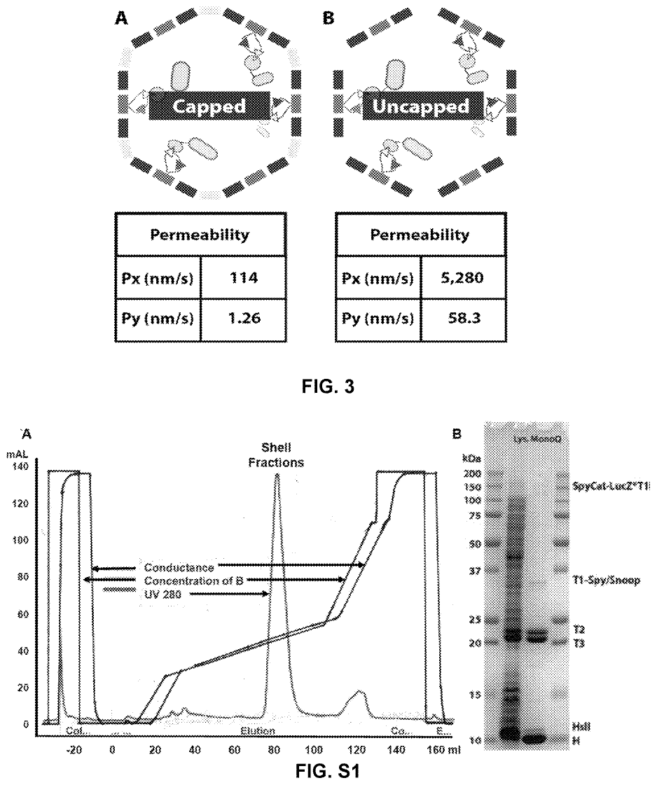

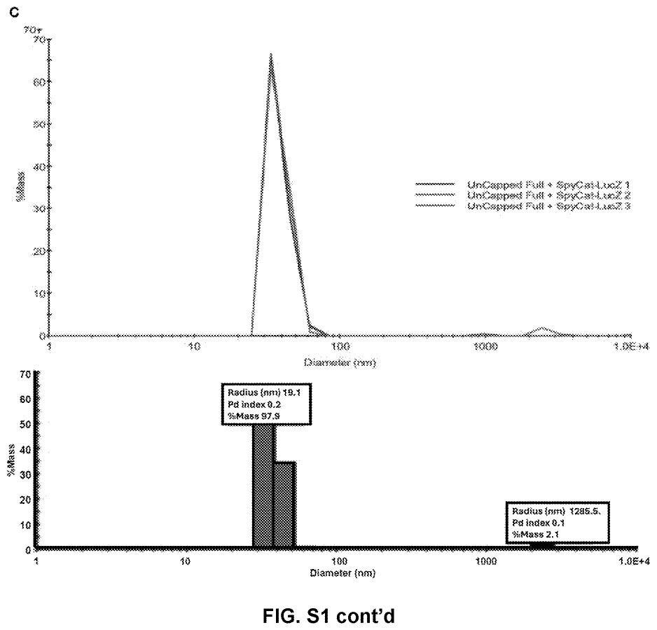

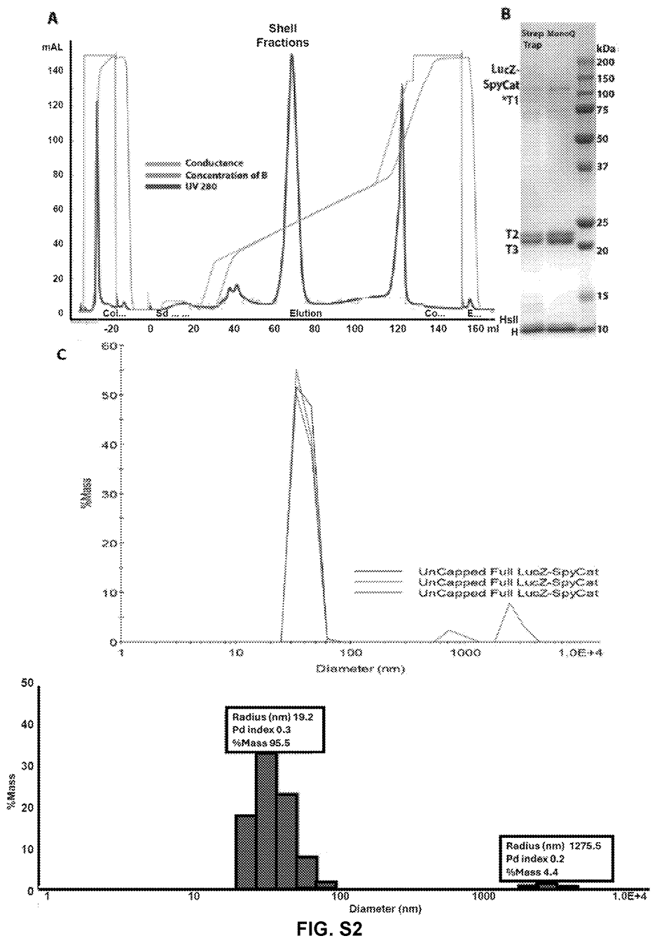

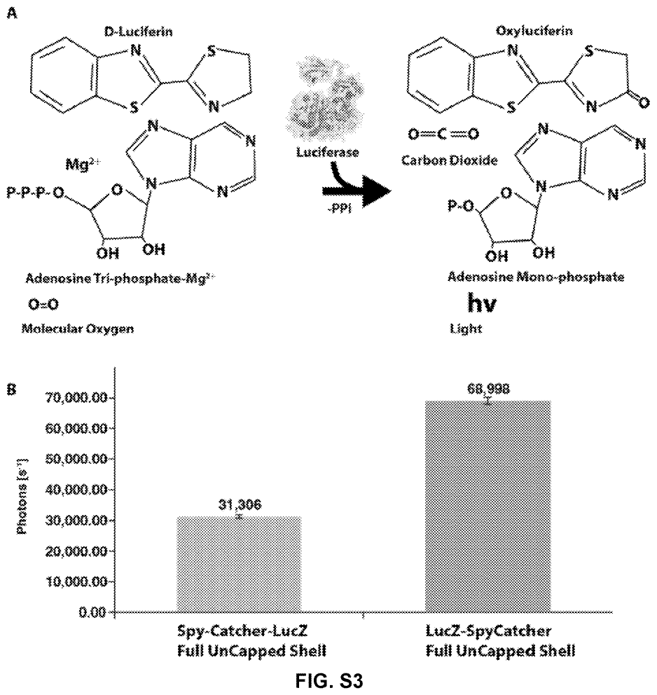

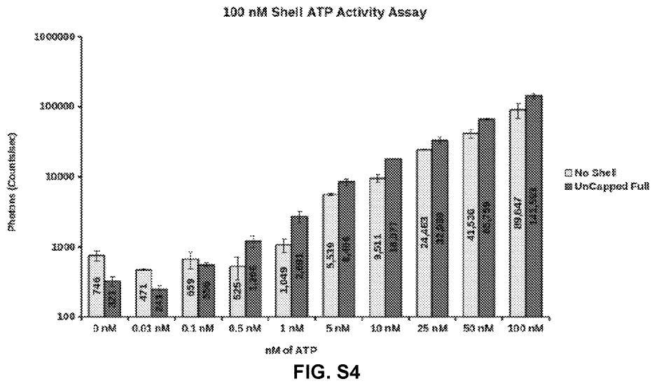

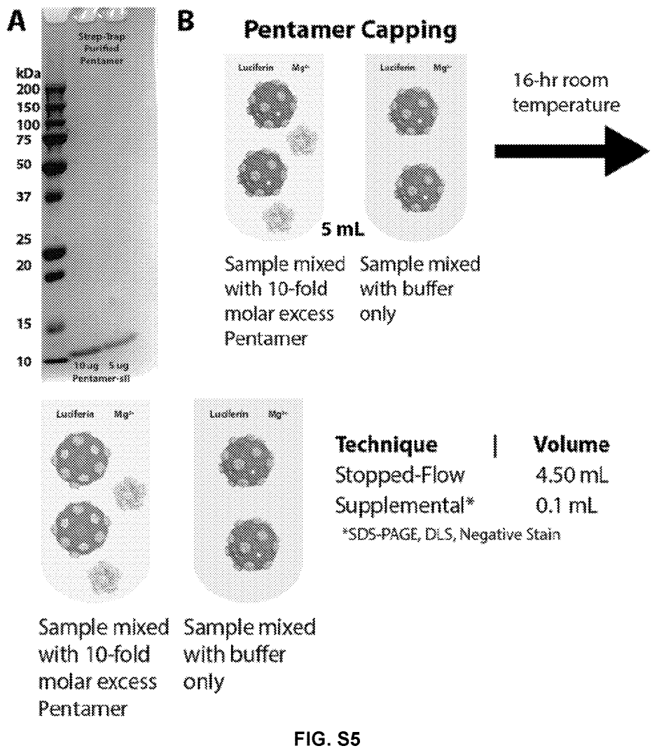

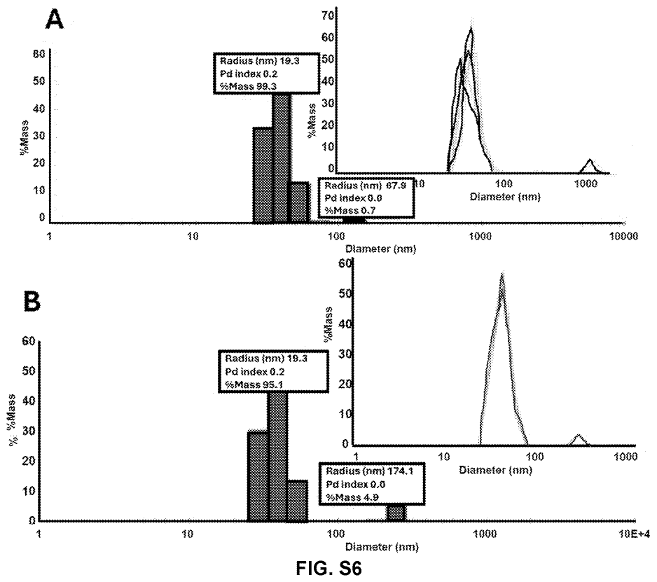

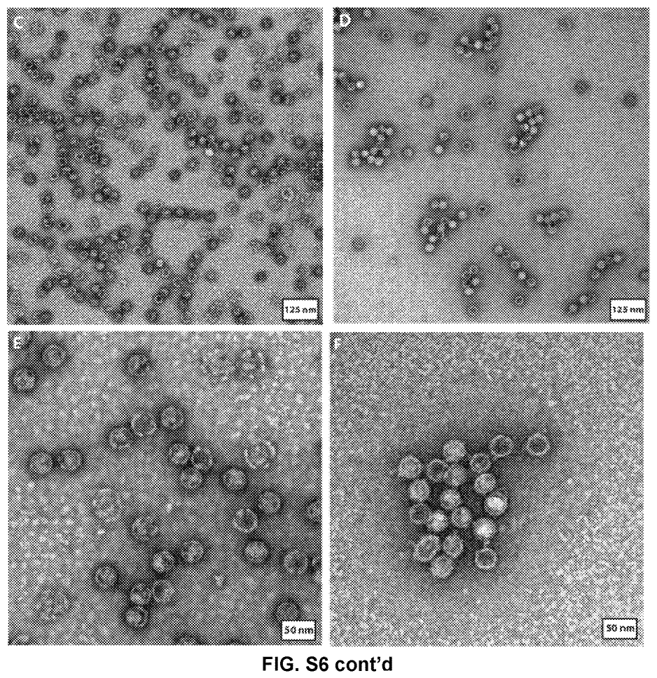

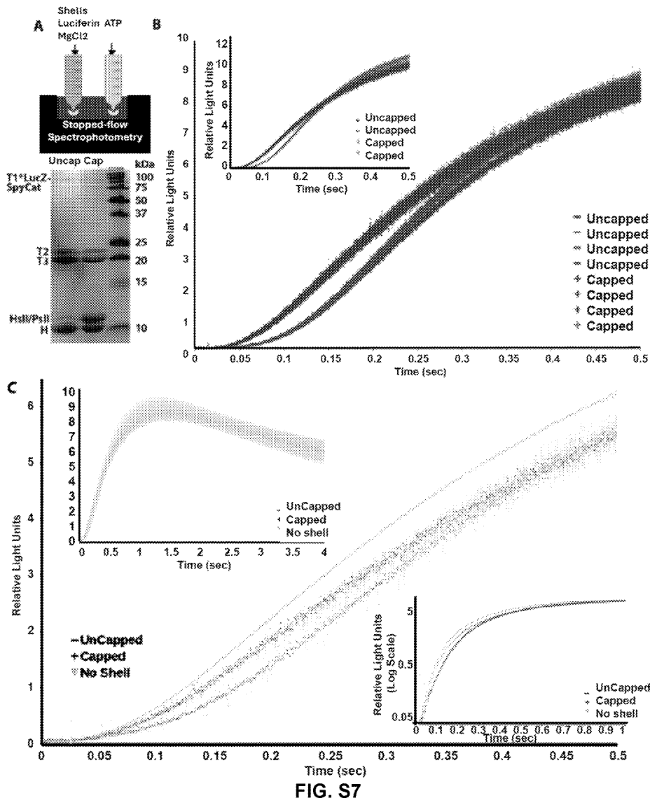

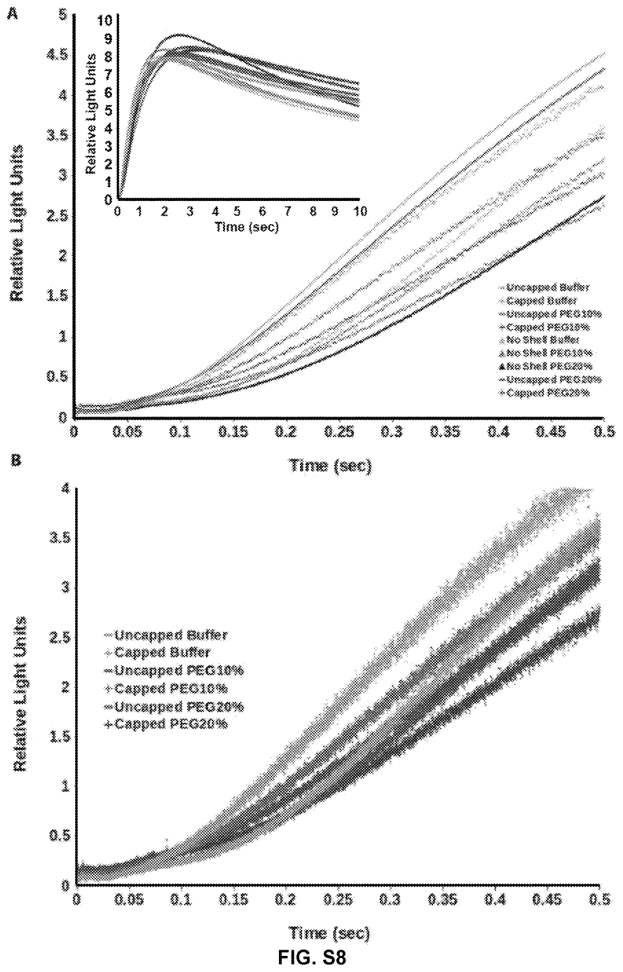

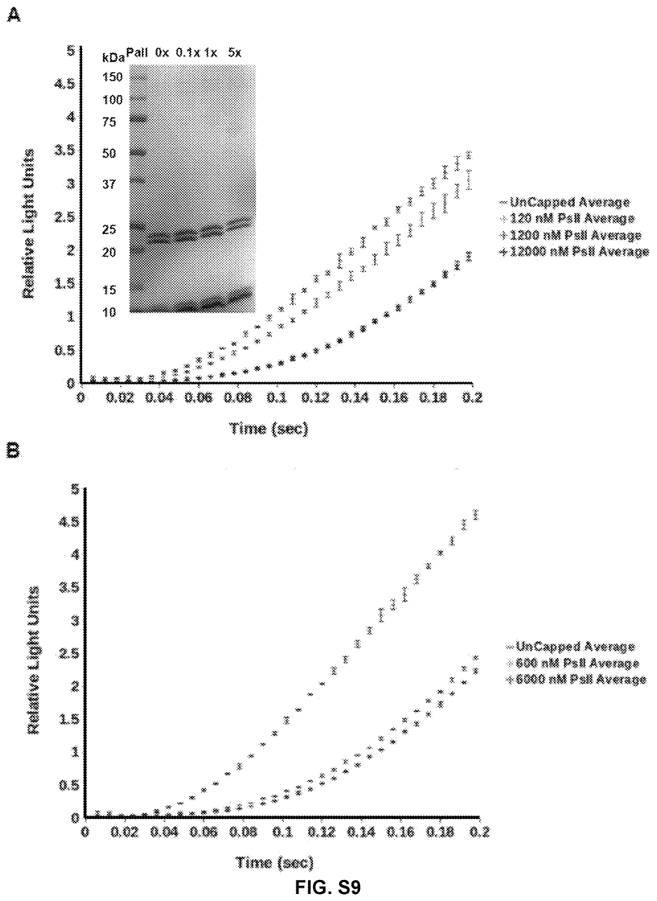

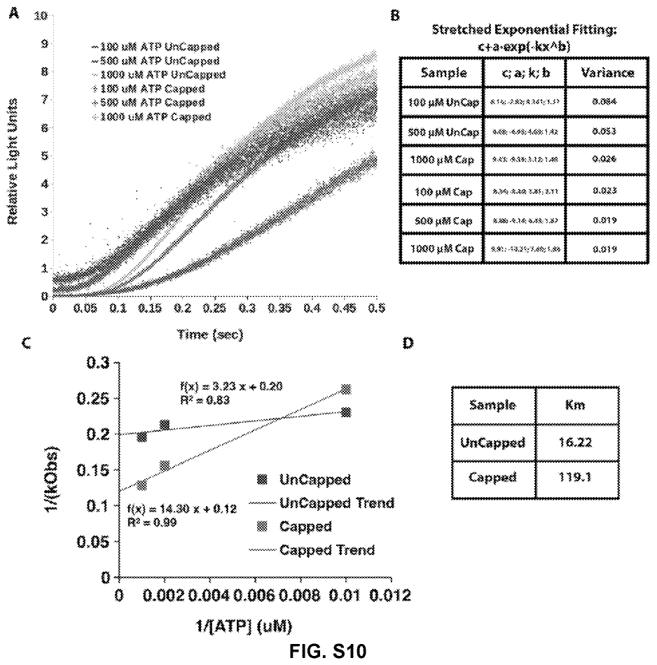

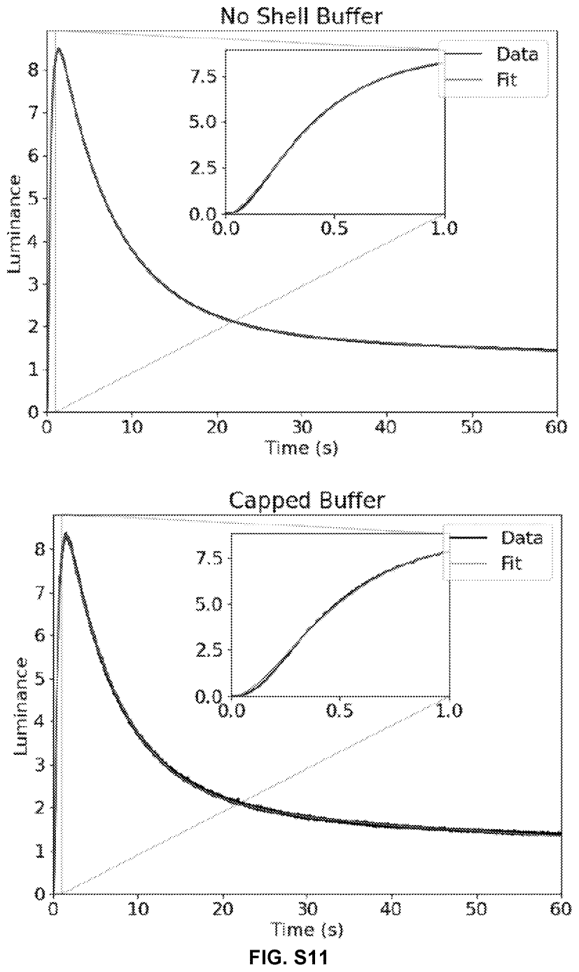

We aggregate comparable datasets together to create an unified fit (Table S2). FIG. 3 summarizes the key findings of this analysis including the permeability coefficient Px, which is a measure for how much the BMC shell represents a diffusion barrier to the rate limiting substrate. While this substrate could be ATP, luciferin or O₂, it is likely that it is ATP, as it is the largest substrate molecule and it was absent in the samples initially in the stopped-flow measurements. Py is the permeability coefficient for the intermediate D-luciferyl-AMP. Py is much smaller than Px, indicating that the BMC shell permits an ATP influx about two orders of magnitude greater than an efflux of the intermediate D-luciferyl-AMP. As can be seen from the data in Supplemental Table 2, the kinetic parameters appear sensitive to solution conditions. However, as the trends in fit parameters are not consistent as the PEG concentration is increased, our interpretation is that the parameters are simply difficult to fit consistently. We see this in evidence again in quantities like the acceleration factor of capped and uncapped shell permeabilities, which is effectively unchanged across solution conditions when the buffer parameters are used to seed the parameters in other solution conditions (Table S2). Once one solution is found, our fitting routines have great difficulty finding dramatically different solutions to the differential equations. Fitting caveats aside, we see that smaller molecules (ATP or luciferin) permeate more quickly than the larger intermediate product and that uncapped shells have faster permeation overall compared to capped shells. The permeability rates calculated appear congruent with stop-flow kinetic measurements (FIG. 2) that qualitatively suggested differences to shell permeability: the luminescence response is fastest without a shell, then if the shell is uncapped, then finally if a shell is capped, representing a growing barrier to molecular access to the shell's interior with enzymes.

Within our model, we assumed that uncapped shell permeation was a consistent multiplicative factor higher than the capped shell permeability coefficient. As the model evolved, and depending on how much data was considered during fitting, this value ranged from around 50 to up to 10,000. The unified fit accounting for as much data as possible supports estimates at the lower end of that range, but we again highlight the uncertainty that is possible. A 50× speedup may however be the most plausible. The pentameric vertices account for only about 7% of the total surface area for an intact 40 nm shell. If this shell reflects about 1200 molecules back into solution for each molecule that permeates, as recently estimated for other small molecules using direct simulation²⁴, a molecule that hits the uncovered 7% of the total surface area would be about 1200 times more likely to permeate through that uncapped space. This simple model implies that the overall rate of permeation would increase by about a factor of 85 in uncapped shells (1200*0.07), which is reasonably close to the 50× rate enhancement determined from the fit.

### Discussion

Our measured values for a BMC shell's permeability (ranging from 10⁻⁷ to 10⁻⁴ cm/s) appear similar in magnitude to permeability values observed for passive transport of small-molecules across lipid bilayers⁵⁸. Based on existing lipid membrane data⁵⁹, calculated permeabilities at this level could support robust small-molecule flux to metabolic pathways encapsulated inside BMC shells. We anticipate that many of the same molecular permeability principles at play in lipid membrane bound compartments may also apply to BMC shells in both native and engineered examples.

For instance, at steady state, the flux across the shell must be identical to catalytic turnover for any encapsulated enzymes, such as the LucZ trapped here. This means that the number of enzyme turnovers is bounded from above by the inbound flux, which is related to the concentration difference and the permeability (Flux=Permeability*Surface Area*ΔC). As a corollary, the concentration inside the shell must be less than the concentration outside the shell in order for substrates to diffuse inside via a positive AC. As a result, catalysis is always slower when a barrier is present with a one enzyme reaction, such as the LucZ system constructed here. This concept appears clear when comparing between capped, uncapped, and no shell conditions (FIG. 2), as luminescence increases fastest when no shell is present. Considering that we can measure luciferase activity encapsulated within a BMC shell over many minutes, it is very likely that not only ATP, but also luciferin and O₂ are able to cross the shell, as these substrates would otherwise be quickly exhausted inside the 40 nm cavity. The permeability coefficients of ATP and D-luciferyl-AMP across the BMC-shell are roughly two orders of magnitude apart, which could be explained by differences to molecular size and charge, indicating that larger and more negatively charged molecules diffuse at a lower rate across a BMC shell.

It is instructive to quantify the molecular fluxes that might be present. For the LucZ system, we can assume a kcat of approximately 1 s⁻¹. Thus to saturate the enzyme, if we assume that the average shell under study here (approximated as a sphere with 19 nm radius) has 5 copies of LucZ, the concentration difference that would saturate the encapsulated enzymes is only 320 μmol/L. Thus, we would expect just a 320 μmol/L concentration difference created by the enzymatic activity inside a shell. Since the concentration outside the shell is much higher in our conditions (in the mmol/L regime) the concentration inside versus outside the BMC shell is difficult to distinguish in our stopped flow experiments. If we use the uncapped permeability, which is about 50× larger, the concentration difference required to saturate the encapsulated enzymes would be 50× smaller.

Under what circumstances might the increased permeability of uncapped shells be advantageous? For single enzyme reactions, uncapping the shells lowers the concentration difference between inside and outside the shell, potentially accelerating kinetics for enzymes that are diffusion limited. We also envision scenarios where catalysis is accelerated by uncapping the shell if a product or reactant is too large to fit through a sealed BMC shell. For multi-enzyme pathways encapsulated within a single BMC, the increased permeability associated with an uncapped shell may be detrimental to the overall catalytic efficiency, as the built up concentration of the product for the first reaction would slip away through the vertex gaps in the shell. If, however, the gaps make up a significantly smaller percentage of the surface area of the BMC shell (such as in larger BMCs similar in size to beta-carboxysomes) the impact of reactants slipping away would be reduced. Additionally, as shown in FIG. S9, BMC shells can also exist in incomplete capping states that have reaction kinetics intermediate to an uncapped or capped system. Incomplete capping in these ways opens the possibility for larger substrates, repair enzymes, or chaperones to exchange across the shell, while activity reduction from small-molecules diffusing out of the shell is minimized.

Accordingly, sealed shells appear to cater best to multi-step pathways. When encapsulating a multi-step pathway, any step after the first one will theoretically have higher enzymatic activity as the intermediate concentrations will be higher inside than outside. Thus, overall performance of a metabolic pathway may increase for particularly slow enzymes requiring a high substrate concentration to work efficiently, as long as the enzymatic step before is also encapsulated and not significantly impacted by the BMC shell diffusion barrier. Regarding this barrier, in general, the first substrate should be able to diffuse inwards as freely as possible to not negatively impact the first catalytic step. However, retention of intermediates formed during reaction should remain inside to benefit downstream enzymatic reactions—this requires the BMC shell to represent a large diffusion barrier in this instance. In fact, this suggested design principle appears observed in the case for BMCs called carboxysomes. These systems encapsulate the catalytically fast carbonic anhydrase delivering the intermediate CO₂ to notoriously catalytically slow and error prone enzyme RuBisCO¹⁷. Because both the permeability properties of the BMC shell towards the first substrate and downstream intermediates might not be completely independently engineerable, compromise appears necessary.

BMCs occur in nearly 70 different varieties of cargo-containing cores and are theoretically built from compositions of nearly 40,000 unique shell proteins^{2,60} Research suggests that cyanobacterial carboxysomes alter compositions of a BMC shell under environmental change, and it is hypothesized that these alterations regulate molecular permeability^{38,40,41}. The detailed influence of a BMC's specific shell protein composition on permeability (versus a dramatic global composition alteration shown here by completely omitting a shell protein creating vertex vacancies) remains to be quantitatively experimentally measured. Growth in computing power continues to push the resolution and timescale at which BMC shells are simulated, even in the presence of metabolites and enzymatic cargos²⁴.

Ultimately, general adoption of our described approach comprising engineering permeability reporters inside BMC shell systems subsequently analyzable by non-invasive dynamical measurements will enable the design-build-test-learn cycle of engineering custom nanoreactors built from BMC shells. Of consideration in this effort is our approximately 8-fold measured change from reported literature of LucZ Km for ATP when in capped configuration⁵⁷. This supports altering a shell's small-molecule permeability rate by including capping pentamers is an important design consideration for how molecularly crowded, sub-diffusive environments impact enzyme activity and/or kinetics 61,62. Additionally, in this effort to describe shell permeability we observed in crowded conditions that encapsulation within a shell prevented activity loss compared to the enzymes free in solution. This is corollary for the design principle in which enzymatic compartmentalization and/or scaffolding improves an enzyme's resilience to crowded and destabilizing environments⁶³.

### MATERIALS AND METHODS

### Molecular Biology, Cell Growth, and Protein Purification

Plasmids were constructed with isothermal DNA assembly from purchased synthetic DNA (IDT) and PCR amplified genes. Table 1 lists the plasmids used in this study. About 50 ng of shell and LucZ cargo plasmids were co-transformed into E. coli BL21 T7 Express (NEB) and selected for on double-resistance LB antibiotic plates. A single colony was grown in 100 mL of antibiotic supplemented LB. A 1:100 dilution of the overnight culture seeded 1 L cultures for protein purification. After growth at 37° C. until OD600 0.8, a twenty minute ice incubation cooled the cultures and then 100 uM IPTG and 50 ng/uL ATc was added to induce protein production. Growth proceeded overnight at 24 degC. 4500×g centrifugation at 4 degC was used to harvest the cells from the LB, and a French Press (about 1250 PSI) (in the presence of a pinch lysozyme, 6 uL Dr. Nuclease, and about 3 mg of DNAase per 20 g of cell paste) ruptured samples in a 50 mM Tris-HCl, 200 mM NaCl pH 8.0 buffer. Lysates were clarified at 27,000×g for 30 minutes. Lysates were stacked on top of a 30% sucrose layer in lysis buffer and ultracentrifugation at 40,000 RPM in a 70Ti rotor for a minimum of 16-hours at 4 degC separated shells from other cellular materials. Glassy ultracentrifugation pellets were resuspended in lysis buffer, mixed until particles dissolved, centrifuged at 14,000×g for 5 minutes to remove insoluble matter, then applied to a 5 mL StrepTrap HP column pre-equilibrated in cold lysis buffer with a syringe pump. Five column volumes of cold lysis buffer were applied to wash away non-specifically bound molecules. 2 or 3 column volumes of 20 mM Tris-HCl, 50 mM NaCl, pH 8.0 buffer with 2.5 mM D-Thiobiotn eluted the sample. Eluant was centrifuged at 14,000×g for 5 minutes at 4 degC and applied with SuperLoop to a 5 mL MonoQ 10/100 GL column at 3 mL/min in 20 mM Tris-HCl, 50 mM NaCl, pH 8.0. A gradient with applied 20 mM Tris-HCl, 1000 mM NaCl, pH 8.0 and the approximately 35%-40% high salt fractions were pooled. 4,200×g centrifugation through a 100 kDa amicon ultrafilter concentrated samples and exchanged shells to lysis buffer. Sample protein purity was analyzed by 20% SDS-PAGE gels. Samples were stored at 4 degC with 0.01% sodium azide added as a preservative. Luciferase activity and shell sample monodispersity was stable for months in this solution (data not shown).

### Luminescence Measurement of Luciferase Reaction

Protein concentration was measured using a NanoDrop Uv-Vis (Thermo Fisher Scientific Inc., Waltham, MA) in E1% mode. Specified ug of protein was added to a black skirted and non-treated 96-well plate (Thermo Fisher Scientific Inc., Waltham, MA). Final reaction volumes were 100-200 uL of lysis buffer with a final concentration of 1 mM luciferin and 1 mM MgCl₂ in the reaction was used. A Tecan Spark microplate reader in luminescence mode was used to measure the luciferase reaction. A specified concentration of ATP either was manually added with a pipette or injected using the Tecan Spark Injector module. Luminescence was recorded with 100 ms integration time and three measurements with two second delays per well.

### Dynamic Light Scattering

A Wyatt DynaPro NanoStar DLS measured protein size in solution. Before measurement, samples were adjusted to 1 mg-mL and centrifuged for 14,000×g for 5 minutes. A 4 uL sample was loaded in the quartz cuvette and data was collected with an acquisition time of four seconds for 25 measurements each per sample with three technical replicates total. Measurements were individually filtered for a baseline value of >1.0 and a sum of squares value of <35.0.

### Transmission Electron Microscopy

Purified samples at 1 mg/mL were diluted to 0.2 mg/mL in water and a 5 uL drop were applied to a 150-mesh copper grid (Electron Microscopy Sciences, Hatfield, USA) on parafilm for 30 seconds. The drop was gently wicked away and stained with a 5 uL droplet of 1% uranyl acetate for 15 seconds (negative stain). Excess uranyl acetate liquid was gently wicked away. Grids were imaged on a JEM-1400 Flash (Jeol, Nieuw-Vennep, Tokyo) TEM at an accelerating voltage of 100 kV.

### Stopped Flow Spectrophotometry

An Applied Photophysics SX20 stopped flow spectrophotometry recorded shell containing luciferase kinetics. Fluorescence mode on the SX20 was used, but the light-source was left off for these reactions in order to collect the light produced by luciferase reaction. After washing the syringes and driving lysis buffer through the mixing chamber, the sample size was set to 10,000 and individual sample syringes were loaded with room temperature solutions. Inlet A contained the solution of shells pre-incubated with MgCl₂ and luciferin substrate in lysis buffer, while Inlet B contained the indicated concentration of ATP in lysis buffer. A collection time-period was set (between 0.125 and 60 seconds) and the drive volume was estimated at 40 uL per syringe. After 10 manual drives to flush the system with a sample, a kinetic spectrum was acquired. Each acquisition obtained five kinetic trace measurements. These traces were then averaged with the Pro Data Viewer SX20 software and exported as a CSV file.

### Data Analysis and Figure Preparation

Open Office Calc (Apache) was used to organize data and prepare graphs. GNU Image Manipulation Program (The GIMP Development Team) and FIJI⁶⁴ were used to adjust the brightness and contrast of images. PyMol (The PyMOL Molecular Graphics System, Version 3.0 Schrödinger, LLC) created protein representations. The ColabFold server using Alphafold2 created the Luciferase and SpyCatcher fusion protein structural model⁶⁵. Adobe Illustrator (Adobe Inc.) was used to construct figure panels.

### Permeability Calculations

A set of coupled differential equations was devised as a model to represent how concentrations vary as a function of time, with the complete derivation presented as Supporting Information. The coupled differential equations that result are similar to prior work by Dale et al ^{54,55} The coupled differential equations were solved numerically through the odefit routine in Scipy⁶⁶. The residual between the solution to the differential equation and the measured luminances from stopped-flow calculations were minimized using the default Levenberg-Marquardt least-squares algorithm implemented in Imfit version 1.2.2 (webpage for: zenodo.org/doi/10.5281/zenodo.598352,⁶⁷. The permeabilities are a model parameter and are read directly from the parameters that minimize the residuals. The code to parse the measured luminescence and implement the fit is available from Github (DOI: 10.5281/zenodo. 10963263).

TABLE S1
Protein Expression Vectors Used In This Study.

| | | | | | Size of Gene |
|---|---|---|---|---|---|
| | | | | Description of | Product(s) |
| Stock | Insert | Backbone | Ab. | Gene Product(s) | (kDa) |
| pEJY066 | H. och 5814 | pBbE2k | KAN | Fusion product of H.och BMC-P to | 11 |
| | BMC-P StrepII | | | StrepII-tag | |
| pEJY107 | H. och BMC | pBbE7a | AMP | Uncapped BMC shell operon with | 10.1; 29.2; 21.9; |
| | Uncapped Shell | | | BMC-H, BMC-T1 with a His-Tag | 22.9; 11.4 |
| | with Covalent | | | C-terminus and a | |
| | Lumen Targeting | | | SpyTag/Snoop Tag loop Insertion, | |
| | and Purification | | | BMC-T3, BMC-T2, and BMC-H with | |
| | Tags | | | a C-terminal Strep-tag | |
| pEJY137 | LucZ- | pBbA2c | CAM | LucZ from Nipponoluciola cruciata | 69.4 |
| | SpyCatcher01 | | | tagged with SpyCatcher01 | |
| PEJY138 | SpyCatcher01- | pBbA2c | CAM | SpyCatcher01 tagged LucZ from | 69.4 |
| | LucZ | | | Nipponoluciola cruciata | |
| pEJY153 | His-LucZ- | pBbA2c | CAM | Fusion protein of His-tagged LucZ | 70.4 |
| | SpyCatcher01 | | | from Nipponoluciola cruciata to | |
| | | | | SpyCatcher01 | |

TABLE S2
Coding Sequences of Expression Vectors. “H. och 5814 BMC-P
StrepII” is SEQ ID NO: 1. “H. och BMC Uncapped Shell with Covalent Lumen Targeting and
Purification Tags” is SEQ ID NO: 2. “LucZSpyCatcher01” is SEQ ID NO: 3. “SpyCatcher01-
LucZ” is SEQ ID NO: 4. “His-LucZSpyCatcher01” is SEQ ID NO: 5.

| Insert | Sequence |
|---|---|
| H. och 5814 | ATGGTCGTGGGTAAAGTCGTGGGTACGGTGGTGGCGAGCCGCAAAGAACCGCGCATTGAAGGTCTGAGCCTGGTGCTGGTCCGTGCCTGCGATCCGG |
| BMC-P StrepII | ACGGTACCCCGACGGGTGGTGCAGTGGTTTGTGCAGATGCAGTGGGTGCAGGTGTTGGTGAAGTCGTGCTGTATGCGAGTGGCAGCTCTGCCCGTCA |
| | GACCGAAGTCACGAACAATCGCCCGGTTGATGCAACCATTATGGCTATCGTTGACCTGGTCGAAATGGGCGGTGATGTGCGTTTTCGCAAAGACGGCAG |
| | CTGGAGTCATCCTCAGTTTGAAAAATAA |
| H. och BMC | [symbol] |
| Uncapped | [symbol] |
| Shell with | [symbol] |
| Covalent | [symbol] |
| Lumen | [symbol] |
| Targeting and | [symbol] |
| Purification | [symbol] |
| Tags | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | GGGGGCAGCTGGAGTCATCCTCAGTTTGAAAAATAA |
| LucZ- | [symbol] |
| SpyCatcher01 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| SpyCatcher01- | [symbol] |
| LucZ | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| His-LucZ- | [symbol] |
| SpyCatcher01 | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |
| | [symbol] |

[symbol]

indicates data missing or illegible when filed

Differential Equations for Luciferase Activity without Shells

Luciferase is well studied, with well worked out biochemistry. The overall bioluminescent reaction catalyzed by firefly luciferase is:

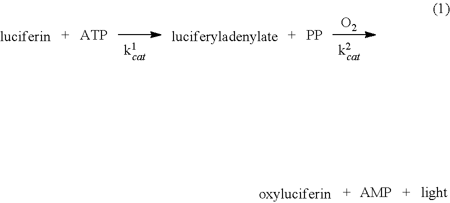

Crucially, the reactions are relatively slow, and luciferase can be inhibited by its products, with both luciferyl adenylate and oxyluciferin having measurable inhibitive effects on luciferase. Thus, unbinding of intermediate products needs to be considered when quantifying luciferase activity by counting photons. Calling the limiting reactant quantity between luciferin and ATP x, luciferyl adenylate y, and oxyluciferin z (and the appropriate enzyme-bound quantities represented as xE and similar), we can solve for the relative concentrations over time within the stopped flow apparatus through the following coupled differential equations:

$$\frac{dx}{dt} = - {k}_{on}^{x} x \cdot E + {k}_{off}^{x} xE \qquad ( 2 )$$

$$\frac{dxE}{dt} = {k}_{on}^{x} x \cdot E - {k}_{off}^{x} xE - {k}_{cat}^{1} xE$$

$$\frac{dy}{dt} = - {k}_{on}^{y} y \cdot E + {k}_{off}^{y} yE$$

$$\frac{dyE}{dt} = {k}_{on}^{y} y \cdot E - {k}_{off}^{y} yE + {k}_{cat}^{1} xE - {k}_{cat}^{2} yE$$

$$\frac{dz}{dt} = - {k}_{on}^{z} z \cdot E + {k}_{off}^{z} zE$$

$$\frac{dzE}{dt} = {k}_{on}^{z} z \cdot E - {k}_{off}^{z} zE + {k}_{cat}^{2} yE$$

$$\frac{dE}{dt} = - {k}_{on}^{x} x \cdot E + {k}_{off}^{x} xE - {k}_{on}^{y} y \cdot E + {k}_{off}^{y} yE - {k}_{on}^{z} z \cdot E + {k}_{off}^{z} zE$$

In these equations, kon is the rate of association between the enzyme and substrate, while k_{off} describes the dissociation process. The association and dissociation rates are superscripted by the species that is associating to disambiguate between the different rate constants. The catalytic rates (k_{cat}) for the two sub-reactions from 1 appear as terms to push the reaction forward. The light emitted (λ) during this process is given by:

$$λ = {Mk}_{cat}^{2} yE \qquad ( 3 )$$

The proportionality constant M in Eq. 3 takes into account that not all photons will be captured by the detector in the stopped flow setup, but otherwise simply measures the flux going through the second reaction in Eq. 1. Using curve-fitting approaches from scipy, we can readily find solutions to the differential equation where the light emitted closely matches the model created by Eqs. 2 and 3. The initial conditions are that at time t=0 there exists freely floating enzyme (E₀) and all of the substrate and product is initially in x, so that x(0)=x0 and that y(0)=z(0)=xE(0)=yE(0)=zE(0)=0.

As can be seen from FIG. S11, this model works really well at the level of fitting individual lines. However, if the fits are made independently, they will predict dissimilar kinetic parameters such as

$${K}_{m} = \frac{{k}_{off} + {k}_{cat}}{{k}_{\text{?}}} and {K}_{d} = \frac{{k}_{off}}{{k}_{on}} .$$

$$\text{?} \text{indicates text missing or illegible when filed}$$

While small variations in experimental conditions are expected, fundamental enzyme thermodynamics should remain unchanged. Since Kd in particular is related to the free energy difference between the bound and unbound states, it should be consistent across fits. We can rewrite Eq. 2 using Kₘ and K_{d} definitions to clarify how many parameters can be independently fit simultaneously.

$$\frac{dx}{dt} = {k}_{on}^{x} ( {K}_{d}^{x} xE - x \cdot E ) \qquad ( 4 )$$

$$\frac{dxE}{dt} = {k}_{on}^{x} ( x \cdot E - {K}_{m}^{x} xE )$$

$$\frac{dy}{dt} = {k}_{on}^{y} ( {K}_{d}^{y} yE - y \cdot E )$$

$$\frac{dyE}{dt} = {k}_{on}^{y} ( y \cdot E - {K}_{m}^{y} yE ) + {k}_{on}^{x} ( {K}_{m}^{x} - {K}_{d}^{z} ) xE$$

$$\frac{dz}{dt} = {k}_{on}^{z} ( {K}_{d}^{z} zE - z \cdot E )$$

$$\frac{dzE}{dt} = {k}_{on}^{z} ( z \cdot E - {K}_{d}^{z} zE ) + {k}_{on}^{y} ( {K}_{m}^{y} - {K}_{d}^{y} ) yE$$

$$\frac{dE}{dt} = {k}_{on}^{x} ( {K}_{d}^{x} xE - x \cdot E ) + {k}_{on}^{y} ( {K}_{d}^{y} yE - y \cdot E ) + {k}_{on}^{z} ( {K}_{d}^{z} zE - z \cdot E )$$

Eq. 4 highlights that there are actually only a few key parameters. kon rates should be sensitive to the solvent environment, as well as the size of the substrate, since the on rate measures of how quickly substrate and enzyme can come together in solution. In particular, adding PEG should vary kon rates proportional to the diffusion coefficient modification that results. K_{d} and Kₘ are thermodynamic quantities that should be independent of a specific fit, since these are all taken at similar ionic strengths, temperatures, and pH. The initial x₀ and E₀ should vary slightly depending on typical experimental variation, and the proportionality constant M should vary directly with E₀, as more enzyme present should emit more light.

Using the data collected where no shell is present, we can fit the existing luminance trends globally using tools like Imfit written for python, with only the initial enzyme and substrate loading left as free parameters.

Adding Permeability into the Model

Where microcompartments change the outlook presented in Eq. 4 is by creating one more barrier that must be crossed. Fick's law tells us that the flux (J) going across a barrier is proportional to the concentration difference across the barrier (ΔC) as well as a permeability coefficient (P) and the surface area of the barrier (A):

$$J = PA Δ C \qquad ( 5 )$$

So the action of encapsulating luciferase inside a capped or uncapped microcompartment shell adds a few extra terms to Eq. 4 in order to describe the movement of molecules into or out of the shells. This takes a little bit of extra book-keeping, since unlike the case where there is no shell, the concentration change needs to account for the difference in volume between the outside and inside of the shell. The number of molecules entering the volume is given by Eq. 5. The rate of concentration change for species “s” moving from compartment A to compartment B is in general related to the flux and the volume of the compartments.

$${\frac{{ds}_{A}}{dt}}_{A \to B} = - \frac{{J}_{A \to B}}{{V}_{A}} = - {P}_{s} \frac{A}{{V}_{A}} ( {s}_{A} - {s}_{B} ) \qquad ( 6 )$$

$${\frac{{ds}_{B}}{dt}}_{A \to B} = \frac{{J}_{A \to B}}{{V}_{B}} = {P}_{s} \frac{A}{{V}_{B}} ( {s}_{A} - {s}_{B} ) = {RP}_{s} \frac{A}{{V}_{A}} ( {s}_{A} - {s}_{B} )$$

In Eq. 6, we define

$$R {=}_{\text{?}} \frac{{V}_{a}}{{V}_{b}} .$$

$$\text{?} \text{indicates text missing or illegible when filed}$$

That way, we are left with a single expression

$$\frac{A}{{V}_{a}}$$

that appears both top and bottom, and is strictly related to the geometry for compartment A. Lets assume that compartment A is the inside of the shell so that compartment B is the region outside the shell. Let us further assume that the shell is spherical, which is close for the icosahedral symmetry present in the shell. The ratio between the surface area (4πr²) and the interior volume of a sphere

$$( \frac{4}{3} π {r}^{3} ) ,$$

regardless of the number of spheres. Since the BMC has a 38 nm diameter, we can eliminate one unknown from the system of equations in Eq. 6 by using this ratio

$$( \frac{3}{r} = \frac{3}{19 nm} ≃ 0.158 nm - 1 ) .$$

In our measurements, the volume outside of the shell (Vo) should be much larger than the total shell interior volume (Vi), so R is anticipated to be small. With these definitions in mind, we add terms to Eq. 4 to account for the shell permeability.

$$\frac{{dx}_{e}}{dt} = {RP}_{x} \frac{3}{19 nm} ( x - {x}_{o} ) \qquad ( 7 )$$

$$\frac{dx}{dt} = {k}_{on}^{x} ( {K}_{d}^{x} xE - x \cdot E ) + {P}_{x} \frac{3}{19 nm} ( {x}_{o} - x )$$

$$\frac{dxE}{dt} = {k}_{on}^{x} ( x \cdot E - {K}_{m}^{x} xE )$$

$$\frac{{dy}_{e}}{dt} = {RP}_{y} \frac{3}{19 nm} ( y - {y}_{o} )$$

$$\frac{dy}{dt} = {k}_{on}^{y} ( {K}_{d}^{y} yE - y \cdot E ) + {P}_{y} \frac{3}{19 nm} ( {y}_{o} - y )$$

$$\frac{dyE}{dt} = {k}_{on}^{y} ( y \cdot E - {K}_{m}^{y} yE ) + {k}_{on}^{x} ( {K}_{m}^{x} - {K}_{d}^{z} ) xE$$

$$\frac{{dz}_{e}}{dt} = {RP}_{z} \frac{3}{19 nm} ( z - {z}_{o} )$$

$$\frac{dz}{dt} = {k}_{on}^{z} ( {K}_{d}^{z} zE - z \cdot E ) + {P}_{z} \frac{3}{19 nm} ( {z}_{o} - z )$$

$$\frac{dzE}{dt} = {k}_{on}^{z} ( z \cdot E - {K}_{d}^{z} zE ) + {k}_{on}^{y} ( {K}_{m}^{y} - {K}_{d}^{y} ) yE$$

$$\frac{dE}{dt} = {k}_{on}^{x} ( {K}_{d}^{x} xE - x \cdot E ) + {k}_{on}^{y} ( {K}_{d}^{y} yE - y \cdot E ) + {k}_{on}^{z} ( {K}_{d}^{z} zE - z \cdot E )$$

What we find particularly powerful about this formulation is that we know the units for the fitted permeability coefficients. Since the input timeseries is explicitly in seconds, and the scaling factor that comes from the ratio between area and volume is in inverse nanometers, the permeabilities fit are explicitly in nm/s. This is in contrast to the K_{d} and Kₘ constants, which are all observed indirectly through the “M” conversion factor from Eq. 3. We anticipate that the light emitted will be less for a given concentration of reactant inside the shell when compared to an equivalent concentration in solution, since the shell only occupies a fraction of the total volume. Indeed, we reuse the multiplicative factor M from Eq. 3 by including a factor

$$\frac{{V}_{i}}{{V}_{total}} \approx \frac{{V}_{i}}{{V}_{o}} = R$$

when correcting for light intensity.

$$λ = RM ( {K}_{m}^{y} - {K}_{d}^{y} ) yE \qquad ( 8 )$$

We can solve the coupled differential equations from Eq. 7 using all the experimental luminance traces as constraints to fit solutions to the underlying differential equations. Using the initial concentration of reactants as the only free parameter between experimental traces, we can robustly fit shared parameters across multiple timeseries to determine reaction rates, K_{d}, Kₘ, and permeabilities. However, since the units for the reaction rates, Kₘ, and K_{d} are not inherently set by geometry like the permeabilities are, we assign two Kₘ values to anchor the fit so that the remaining optimization parameters have explicit units. Based on prior literature, we set the Kₘ for oxyluciferin to be 14.7 μM, and the Kₘ for L-AMP to be 14.9 μM.1 In practice, the fitting procedure is done iteratively, starting from an initial guess and using Imfit 1.2.2 to optimize first the curves without shells present before using the optimal values from that fit as the starting point for when shells are added.

Another change in practice is that the dependence on the oxyluciferin concentration within the shell (z) is very weakly coupled to other quantities of interest. The measured quantity in experiment is the light emission, which depends strongly on the luciferyl adenylate complex with luciferin (yE, Eq. 8). The oxyluciferin concentration grows as a direct consequence of light emission, but its impact on regulating the emitted light is very weak. Indeed, careful analysis of Eq. 7 highlights that the oxyluciferin concentration (z, red elements in Eq. 7) can only feedback into the differential equations that lead to light emission by binding to free enzyme. As a consequence, directly fit terms like k^{z}ₒₙ, K^{z}_{d} from this weak signal created large uncertainties around these terms, with the optimizer unable to distinguish between many different values. We opted to simplify Eq. 7 and eliminate the z-dependent terms from Eq. 7 that are shown in red. Eliminating these terms only modestly increases the mean squared error, and creates a model where the uncertainties in each term are reasonable.

Since oxyluciferin only connects to the light emitted by binding to free enzyme, we noted that the uncertainties for the fit parameters around z-dependent terms were unusually high, and in many instances were higher than the imputed value. Eliminating oxyluciferin-dependent terms within Eq. 7 only modestly increases the mean squared error, and creates a model where the uncertainties are reasonable.

The code for the fitting, and reading in the experimental data, is provided on github (DOI: 10.5281/zenodo. 10963263).

TABLE S3
Full table of fit parameters.

| | Solution | | |
|---|---|---|---|
| Parameter | Buffer | PEG 10% | PEG 20% |
| Pₓ (nm/s) | 1.14 × 10² | 2.29 × 10² | 3.25 × 10³ |
| P_{y} (nm/s) | 1.26 | 4.48 × 10¹ | 2.3 |
| Uncapped acceleration | 4.63 × 10¹ | 4.63 × 10¹ | 4.63 × 10¹ |
| K_{d}^{x} (μmol L⁻¹) | 3.19 | 2.46 | 6.03 × 10⁻¹ |
| K_{d}^{y} (μmol L⁻¹) | 1.27 × 10¹ | 2.44 | 1.21 × 10¹ |
| Kₘ^{x} (μmol L⁻¹) | 1.47 × 10¹ | 3.31 | 2.64 × 10¹ |
| Kₘ^{y} (μmol L⁻¹) | 1.49 × 10¹ | 6.79 | 1.6 × 10¹ |
| Kₒₙ^{x} (s⁻¹) | 2.36 × 10⁻¹ | 1.97 | 1.81 × 10⁻¹ |
| Kₒₙ^{y} (s⁻¹) | 1.59 × 10⁻¹ | 2.56 × 10⁻¹ | 7.06 × 10⁻² |
| RMSE | 0.20 | 0.06 | 0.16 |

### REFERENCES

[1] Ribeiro, C.; Esteves Da Silva, J. C. G. Photochem Photobiol Sci 2008, 7, 1085-1090.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A fusion protein comprising (1) a bacterial microcompartment (BMC) shell protein comprising one or more subunit, and (2) a first component of a specific-binding pair, operably linked to the BMC shell protein such that the first component faces (i) a lumen (inside) side, or (ii) outside of a BMC shell formed incorporating the fusion protein and the fusion protein does not disrupt or prevent the folding of the BMC shell protein or the ability of the BMC shell protein to integrate with other BMC shell proteins into a BMC shell; wherein the first component is capable of forming a stable or irreversible interaction with a second component of the specific-binding pair; wherein the second component comprises a cargo or compound capable of generating an output directly coupled to the molecular permeability of the BMC shell.

2. A bacterial microcompartment (BMC) or BMC shell comprising the fusion protein of claim 1.

3. A nucleic acid encoding the fusion protein of claim 1.

4. A host cell comprising the nucleic acid encoding the fusion protein of claim 1.


SEQUENCE LISTING (6 sequences)

SEQ ID NO: 1 - DNA, 321 bp, Synthetic construct
  source 1..321: mol_type = other DNA
atggtcctgggtaaagtcgtgggtacggtggtggcgagccgcaaagaaccgcgcattgaaggtctgagcctgctgctggtccgtgcctgcgatccggacggtaccccgacgggtggtgcagtggtttgtgcagatgcagtgggtgcaggtgttggtgaagtcgtgctgtatgcgagtggcagctctgcccgtcagaccgaagtcacgaacaatcgcccggttgatgcaaccattatggctatcgttgacctggtcgaaatgggcggtgatgtgcgttttcgcaaagacggcagctggagtcatcctcagtttgaaaaataa

SEQ ID NO: 2 - DNA, 2845 bp, Synthetic construct
  source 1..2845: mol_type = other DNA
atggcggacgcactgggtatgattgaagttcgtggttttgttggtatggtggaagcggcggatgctatggtgaaagcggctaaagttgaactgattggttatgaaaaaaccggcggtggctacgtgacggcagtggttcgtggtgatgtcgcagcagttaaggcagctaccgaagccggtcagcgtgcagcagaacgtgttggtgaagtcgtggcagttcatgtcatcccgcgtccgcacgtgaacgttgatgcagctctgccgctgggtcgtacgccgggtatggacaaaagcgcgtaatttagagattaaagaggagaaatactagatggaccacgctccggaacgctttgatgcgaccccgccggcaggtgaaccggaccgcccggcactgggtgtgctggaactgacctcaattgctcgtggtatcaccgttgcggatgcggccctgaaacgtgcaccgagtctgctgctgatgtcccgcccggtcagctctggcaagcatctgctgatgatgcgtggccaggtggcagaagttgaagaatcaatgattgcagctcgcgaaatcgctggtgcaggtccgaagccgaaaccaaaacctaagcctaagggtgctcatattgtcatggttgatgcttataagccaactaagggtcctaagccaaagccgaaacccaaaggtggctctggtggaggtggaagtggtggtaaactgggcgatattgaatttattaaagtgaacaaaggaggtagcggcggtggaggttcaggcggaggtgctctgctggatgaactggaactgccgtatgcgcacgaacaactgtggcgctttctggacgcaccggtggttgcagatgcatgggaagaagacaccgaaagcgtcattatcgtggaaaccgcgacggtgtgcgcggccattgatagtgccgacgcagctctgaaaacggcaccggtcgtgctgcgtgatatgcgcctggccattggtatcgctggcaaggcgtttttcaccctgacgggtgaactggcagacgtggaagcggccgcagaagttgtccgtgaacgttgcggtgcacgtctgctggaactggcatgtatcgcacgcccggttgatgaactgcgtggccgcctgtttttcggtggctctggtggacatcatcaccatcaccactaatttagagattaaagaggagaaatactagatggaactgcgtgcttatacggtcctggatgccctgcagccgcaactggtcgcctttctgcaaacggtgtcaacgggtttcatgccgatggaacagcaagcgagcgttctggtcgaaattgcaccgggtatcgctgtcaaccagctgaccgacgcagcactgaaagcaacgcgttgccagccgggtctgcaaattgtggaacgtgcgtatggcctgatcgaaatgcatgatgacgatcagggtcaagttcgtgcagctggtgacgcaatgctggcacacctgggtgcacgtgaagctgatcgtctggcaccgcgtgtggttagctctcagattatcaccggtattgacggccatcagagtcaactgatcaaccgtatgcgccacggtgatatgattcaggcaggccaaacgctgtatatcctggaagttcatccggcaggttacgcagcactggcagctaatgaagccgaaaaagcggccccgattaagctgctggaagtcgtgacctttggtgcattcggtcgtctgtggctgggtggtggtgaagcagaaatcgcagaagcagctcgtgcggcagaaggtgcactggctggtctgtccggccgtgataatcgcggctaatttagagattaaagaggagaaatactagatgtcaatcaccctgcgcacctatatctttctggacgccctgcaaccgcaactggcaaccttcatcggcaaaacggctcgtggcttcctgccggtcccgggtcaggcaagcctgtgggtggaaattgctccgggtattgcgatcaaccgtgtgaccgatgcggccctgaaagctacgaaggtgcagccggcggttcaagtggttgaacgcgcgtatggcctgctggaagttcatcacttcgatcagggcgaagtcctggcagctggtagtaccatcctggacaaactggaagttcgtgaagaaggtcgcctgaagccgcaggtgatgacccatcaaattatccgtgctgttgaagcgtatcagacgcaaattatcaaccgcaatagtcagggcatgatgattctgccgggtgaatccctgtttatcctggaaacccaaccggcaggttacgcagtcctggcagccaatgaagccgaaaaagcagctaacgttcacctggtcaatgtgacgccgtatggcgcattcggtcgtctgtacctggccggctcagaagcagaaattgatgcggccgcagaagctgcggaagccgcaatccgcagcgtttctggtgtcgcgcaggaatcgtttcgtgaccgctaattaagtataagaaggagatatacatatggcggacgcactgggtatgattgaagttcgtggttttgttggtatggtggaagcggcggatgctatggtgaaagcggctaaagttgaactgattggttatgaaaaaaccggcggtggctacgtgacggcagtggttcgtggtgatgtcgcagcagttaaggcagctaccgaagccggtcagcgtgcagcagaacgtgttggtgaagtcgtggcagttcatgtcatcccgcgtccgcacgtgaacgttgatgcagctctgccgctgggtcgtacgccgggtatggacaaaagcgcgggtggcagctggagtcatcctcagtttgaaaaataa

SEQ ID NO: 3 - DNA, 1911 bp, Synthetic construct
  source 1..1911: mol_type = other DNA
atggaaaacatggaaaatgacgagaacatagtggtaggtcctaaaccgttttatccgattgaagaggggtctgccggaactcagcttcgcaagtatatggaaagatatgcgaaattaggagcaatcgcattcacaaatgcagtgactggagtagactatagttatgcggaatatttagagaagagttgctgtctggggaaagcgttacagaactacgggttagtagtagatggcagaattgcgctgtgctcggaaaactgcgaagaatttttcatccccgtcatagcgggccttttcatcggtgtgggtgttgcaccgaccaacgagatttatacgttacgcgaattagtccattctctgggtatttccaaaccaaccattgttttctctagtaagaagggcctggataaagtcataaccgtccaaaaaactgtaacgacgatcaaaactattgtgatcttagattccaaggtagactacagaggttatcagtgccttgatacctttattaaaagaaataccccacctgggttccaggcgtcaagtttcaagaccgttgaagtggaccgcaaggaacaggtagcccttatcatgaactcatcgggatcaacaggtctgccgaaaggagtacaacttacgcacgagaatatcgtgacccggtttagtcatgctcgggacccaatttacggtaatcaagtttcccccggaaccgccgtcttgacggtagtcccgtttcaccacggctttgggatgttcactaccctgggctacttaatatgtggcttccgggtcgtaatgcttaccaaattcgatgaggagaccttcttaaagaccttacaagactataagtgcacgagcgttattttagtgccgacgctttttgccattctgaataaatcagaattattaaacaagtatgacttatcaaaccttgtggaaattgcttctgggggtgctcccttgagtaaggaggtaggcgaagctgtagctcgcagatttaatctgccgggggtgagacaaggttacggacttacggagactacaagcgccattattattacaccagagggagacgacaagcccggtgcgagtggaaaggtggtacccttgtttaaggcaaaagtcattgacctggacaccaagaaatccttagggccgaacagacgcggcgaggtgtgcgtgaaggggcccatgctgatgaaagggtatgtgaataatcccgaggcgacaaaggagttgatcgacgaggaaggttggttacacactggagacattgggtactacgacgaggaaaagcatttcttcatagtcgatcgcttaaaaagcctgattaaatataaaggttatcaagtgccacctgcggaacttgaatcggttctgttacagcacccctctatttttgatgctggtgttgcaggggtgccggaccctgtggcaggtgagttgccaggggccgtcgtggtgttagaaagtgggaaaaatatgaccgaaaaagaggttatggattatgttgcgtctcaagtaagtaatgccaagcggctgcgcggaggagtacggttcgtagacgaggtaccaaaaggtttaacagggaagattgacgggcgcgcgatacgcgaaatacttaaaaagccggtagctaaaatgggtggatcaggtggtagtgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaattaa

SEQ ID NO: 4 - DNA, 1908 bp, Synthetic construct
  source 1..1908: mol_type = other DNA
atggatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggaggtagcggaggaagtgaaaacatggaaaatgacgagaacatagtggtaggtcctaaaccgttttatccgattgaagaggggtctgccggaactcagcttcgcaagtatatggaaagatatgcgaaattaggagcaatcgcattcacaaatgcagtgactggagtagactatagttatgcggaatatttagagaagagttgctgtctggggaaagcgttacagaactacgggttagtagtagatggcagaattgcgctgtgctcggaaaactgcgaagaatttttcatccccgtcatagcgggccttttcatcggtgtgggtgttgcaccgaccaacgagatttatacgttacgcgaattagtccattctctgggtatttccaaaccaaccattgttttctctagtaagaagggcctggataaagtcataaccgtccaaaaaactgtaacgacgatcaaaactattgtgatcttagattccaaggtagactacagaggttatcagtgccttgatacctttattaaaagaaataccccacctgggttccaggcgtcaagtttcaagaccgttgaagtggaccgcaaggaacaggtagcccttatcatgaactcatcgggatcaacaggtctgccgaaaggagtacaacttacgcacgagaatatcgtgacccggtttagtcatgctcgggacccaatttacggtaatcaagtttcccccggaaccgccgtcttgacggtagtcccgtttcaccacggctttgggatgttcactaccctgggctacttaatatgtggcttccgggtcgtaatgcttaccaaattcgatgaggagaccttcttaaagaccttacaagactataagtgcacgagcgttattttagtgccgacgctttttgccattctgaataaatcagaattattaaacaagtatgacttatcaaaccttgtggaaattgcttctgggggtgctcccttgagtaaggaggtaggcgaagctgtagctcgcagatttaatctgccgggggtgagacaaggttacggacttacggagactacaagcgccattattattacaccagagggagacgacaagcccggtgcgagtggaaaggtggtacccttgtttaaggcaaaagtcattgacctggacaccaagaaatccttagggccgaacagacgcggcgaggtgtgcgtgaaggggcccatgctgatgaaagggtatgtgaataatcccgaggcgacaaaggagttgatcgacgaggaaggttggttacacactggagacattgggtactacgacgaggaaaagcatttcttcatagtcgatcgcttaaaaagcctgattaaatataaaggttatcaagtgccacctgcggaacttgaatcggttctgttacagcacccctctatttttgatgctggtgttgcaggggtgccggaccctgtggcaggtgagttgccaggggccgtcgtggtgttagaaagtgggaaaaatatgaccgaaaaagaggttatggattatgttgcgtctcaagtaagtaatgccaagcggctgcgcggaggagtacggttcgtagacgaggtaccaaaaggtttaacagggaagattgacgggcgcgcgatacgcgaaatacttaaaaagccggtagctaaaatg

SEQ ID NO: 5 - DNA, 1914 bp, Synthetic construct
  source 1..1914: mol_type = other DNA
atggaaaacatggaaaatgacgagaacatagtggtaggtcctaaaccgttttatccgattgaagaggggtctgccggaactcagcttcgcaagtatatggaaagatatgcgaaattaggagcaatcgcattcacaaatgcagtgactggagtagactatagttatgcggaatatttagagaagagttgctgtctggggaaagcgttacagaactacgggttagtagtagatggcagaattgcgctgtgctcggaaaactgcgaagaatttttcatccccgtcatagcgggccttttcatcggtgtgggtgttgcaccgaccaacgagatttatacgttacgcgaattagtccattctctgggtatttccaaaccaaccattgttttctctagtaagaagggcctggataaagtcataaccgtccaaaaaactgtaacgacgatcaaaactattgtgatcttagattccaaggtagactacagaggttatcagtgccttgatacctttattaaaagaaataccccacctgggttccaggcgtcaagtttcaagaccgttgaagtggaccgcaaggaacaggtagcccttatcatgaactcatcgggatcaacaggtctgccgaaaggagtacaacttacgcacgagaatatcgtgacccggtttagtcatgctcgggacccaatttacggtaatcaagtttcccccggaaccgccgtcttgacggtagtcccgtttcaccacggctttgggatgttcactaccctgggctacttaatatgtggcttccgggtcgtaatgcttaccaaattcgatgaggagaccttcttaaagaccttacaagactataagtgcacgagcgttattttagtgccgacgctttttgccattctgaataaatcagaattattaaacaagtatgacttatcaaaccttgtggaaattgcttctgggggtgctcccttgagtaaggaggtaggcgaagctgtagctcgcagatttaatctgccgggggtgagacaaggttacggacttacggagactacaagcgccattattattacaccagagggagacgacaagcccggtgcgagtggaaaggtggtacccttgtttaaggcaaaagtcattgacctggacaccaagaaatccttagggccgaacagacgcggcgaggtgtgcgtgaaggggcccatgctgatgaaagggtatgtgaataatcccgaggcgacaaaggagttgatcgacgaggaaggttggttacacactggagacattgggtactacgacgaggaaaagcatttcttcatagtcgatcgcttaaaaagcctgattaaatataaaggttatcaagtgccacctgcggaacttgaatcggttctgttacagcacccctctatttttgatgctggtgttgcaggggtgccggaccctgtggcaggtgagttgccaggggccgtcgtggtgttagaaagtgggaaaaatatgaccgaaaaagaggttatggattatgttgcgtctcaagtaagtaatgccaagcggctgcgcggaggagtacggttcgtagacgaggtaccaaaaggtttaacagggaagattgacgggcgcgcgatacgcgaaatacttaaaaagccggtagctaaaatgggtggatcaggtggtagtgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaattaataa

SEQ ID NO: 6 - protein, 5 aa, Haliangium ochraceum
AGSGA